(19) United States
(12) Patent Application Publication
(10) Pub. No.: US 2026/0242870 A1
(43) Pub. Date: Aug. 20, 2026

(54) SUPER RESOLUTION MICROSCOPIC IMAGING OF T LYMPHOCYTE CLONALITY COMBINING MULTIPLEXED SEQUENTIAL FLUORESCENCE IN SITU HYBRIDIZATION WITH BULK GENOMIC DNA SEQUENCING

(71) Applicant: CALIFORNIA INSTITUTE OF TECHNOLOGY, Pasadena, CA (US)

(72) Inventors: Jonathan J. FOX, Pasadena, CA (US); Michal Polonsky, Pasadena, CA (US); Long Cai, Pasadena, CA (US); Haiyun Liu, Pasadena, CA (US); Chuqi Lu, Pasadena, CA (US)

(21) Appl. No.: 19/536,073

(22) Filed: Feb. 10, 2026

Related U.S. Application Data
(60) Provisional application No. 63/756,535, filed on Feb. 10, 2025.

(51) Int. Cl. C12Q 1/6881; C12Q 1/6837

(52) U.S. Cl. CPC C12Q 1/6881; C12Q 1/6837

(57) ABSTRACT

This disclosure provides methods for imaging of samples for diagnosis of lymphoid neoplasms, lymphocytic autoimmune disease, and other conditions related to clonal expansion of lymphocytes. Using these methods, individual lymphocytes can be imaged in situ to determine tissue-wide clonality based on rearrangement of T cell receptor gene segments. Post hoc bulk sequencing allows for complementarity determining sequences to be mapped back to individual lymphocyte clones in super resolution images. In additional to its clinical applications in histopathology, these methods are also useful for the study of cellular and immunologic processes related to lymphoproliferative and autoimmune disorders.

### PRIORITY

This application claims priority to U.S. Provisional Application No. 63/756,535, filed on Feb. 10, 2025, and entitled “SUPER RESOLUTION MICROSCOPIC IMAGING OF T LYMPHOCYTE CLONALITY COMBINING MULTIPLEXED SEQUENTIAL FLUORESCENCE IN SITU HYBRIDIZATION WITH BULK GENOMIC DNA SEQUENCING.” The foregoing application is incorporated herein by reference in its entirety.

### STATEMENT REGARDING FEDERALLY-SPONSORED RESEARCH

This invention was made with government support under Grant No. 1U01CA294551 awarded by the National Institutes of Health. The government has certain rights in the invention.

### FIELD OF INVENTION

The present disclosure provides methods for barcoding and imaging lymphocyte clonality useful for the evaluation of clinical samples for the diagnosis and management of lymphoid neoplasms and autoimmune diseases.

### BACKGROUND

The evaluation of lymphocyte clonality is a key step in the diagnosis and management of lymphoid neoplasms and lymphocyte-mediated autoimmune diseases. Clinical samples are routinely tested for clonality in patients presenting with suspected lymphocytic leukemias and lymphomas, where positive clonality often serves as confirmation of disease. Of the various methods used by clinical laboratories to evaluate lymphocytic clonality, the most common are flow cytometry, multiplexed polymerase chain reaction (PCR) and next-generation sequencing (NGS). While these methods can identify T cell clones in clinical samples, they may lack spatial information, which is particularly useful in distinguishing pathogenic clones from within polyclonal samples. The present disclosure describes an alternative to PCR and NGS for the detection of lymphocyte gene rearrangements in the evaluation of lymphocytic clonality. In addition, this method allows for NGS to be performed post hoc to map complementarity-determining sequences to individual T cells in super resolution images.

Clonality in the context of lymphocytes refers to the expression of unique T cell receptor (TCR) or immunoglobulin (Ig) proteins by individual T or B lymphocytes, respectively. A considerable amount of variance in these proteins is essential for adaptive immunity, where the immune system must recognize and respond to a vast repertoire of antigens, including proteins of foreign pathogens and tumor neo-antigens. The diversity in antigenic recognition is mainly achieved through somatic recombination of various gene segments during the lymphocytic maturation process. Each mature TCR or Ig consists of a constant (C) segment connected to randomly selected variable (V) and joining (J) segments (some genes, such as TCRβ, have an additional diversity (D) segment between V and J). In a process known as V(D)J recombination, RAG1/2 expressed in maturing lymphocytes recognizes recombination signal sequences flanking randomly selected V, D, and J coding segments, HMG1A/B helps to cleave the selected segments, and they are somatically recombined by non-homologous end joining. Different combinations of V, D, and J segments expressed in individual lymphocytes allow for diversity in antigenic recognition and result in a polyclonal lymphocyte repertoire. Besides V(D)J recombination, additional diversity is generated by nucleotide insertion and deletion at V-D, D-J, and V-J junctional sites. At a structural level, each TCR subunit (α, β, γ, and δ) contains 3 loops which come into contact with the peptide-MHC (pMHC) complex, each known as a complementarity-determining region (CDR). CDR1 and CDR2 are encoded solely by the V segment and bind to conserved α-helices of the MHC. CDR3, meanwhile, is thought to be the most important loop in determining TCR epitope specificity because it is encoded at the hypervariable junction between V and J segments, and it touches the peptide in the pMHC complex. Thus, clonality in the context of T lymphocytes describes groups of T cells sharing specific TCR sequences (αβ or γδ), which is indicative of a shared origin and ability to bind to and recognize a specific epitope or a group of related epitopes.

Identification of clonal expansion is routinely performed in the workup of suspected lymphoproliferative neoplasms. Because the malignancy originates from a single precursor cell with a unique set of gene rearrangements, overrepresentation of a single clone in a clinical sample is suggestive of cancer rather than normal lymphocytic inflammation. Additionally, auto-reactive lymphocyte clones have been implicated in a number of autoimmune diseases; however, testing for clonality is not routinely performed in these cases. Identification and study of lymphocyte clonality in autoimmune diseases may therefore help further our understanding of the underlying pathogenic mechanisms and therapeutic options.

The earliest laboratory tests for lymphoid clonality used restriction enzymes and Southern blotting to identify clones. Multiplexed PCR assays were later developed and standardized for the detection of clonal lymphocyte gene rearrangements, and it continues to be the most widely used test in clinical laboratories. PCR-based assays employ sets of primers against V and J segments to amplify recombined DNA from clinical samples, with the lengths of amplified loci used to distinguish monoclonality from polyclonality using gel electrophoresis. PCR-based approaches are limited, however, by amplicon size. The TRA locus, for example, is too large to amplify by PCR, so clonality in these assays is usually assessed using only TRB and TRG. While PCR is generally sufficient for assessing clonality, it is unable to detect clonality in a subset of lymphomas (~20%) and it provides no information about the identity of the clones or their antigenic targets. More recently, new methods of assessing clonality utilizing NGS have been developed. By directly sequencing rearranged TCR loci, NGS-based assays have the advantages of distinguishing between different clones with similar lengths and defining the peptide sequence of the CDRs in the final TCR, thus providing structural information related to epitope specificity. NGS assays still suffer from the same limitations as PCR concerning amplicon size during library preparation. Thus, prediction of antigen specificity is incomplete due to the lack of available training data for difficult-to-amplify genes. Additionally, neither PCR nor NGS assays currently used in clinical laboratories provide information about the spatial distribution of lymphocyte clones within the tissue, information which may be useful in conjunction with histological analysis for diagnostic purposes.

Recently, NGS using spatially barcoded capture slides has been deployed for localizing TCR clonotypes within tissues, identifying immunological niches, and clonal heterogeneity within sections of tumors. These technologies highlight the utility of preserving spatial information with regard to assessing lymphocyte clonality in the clinic and in research. However, NGS-based spatial methods are limited by the resolution of the capture areas (55 m in Visium), diffusion of transcripts, and bias for poly-adenylated transcripts. Sequential fluorescence in situ hybridization (seqFISH) is an alternative spatial transcriptomic method developed by our lab, which uses multiplexed barcoded oligonucleotide probes, custom microfluidics, and confocal microscopy to image individual transcripts at a subcellular resolution. In this disclosure, a method is described to evaluate lymphocyte clonality in fixed tissue sections using seqFISH. This method utilizes barcoded oligonucleotide probes for the detection of T cell clones using pairing of V and J TCR gene segments in mRNA. After imaging, genomic DNA or mRNA can then be extracted off the slide and sequenced for rearranged TCR loci to allow for mapping of specific CDR3 sequences back to the clones identified by seqFISH in the tissue, thus providing TCR structural data in super resolution transcriptomic seqFISH microscopy.

### SUMMARY

This disclosure provides methods for imaging of samples for the diagnosis of lymphoid neoplasms, lymphocytic autoimmune disease, and other conditions related to clonal expansion of lymphocytes.

In some embodiments, a method is described comprising contacting a cell population with a plurality of primary probes. In certain embodiments, the plurality of primary probes comprises a first primary probe that interacts with a first gene segment. In certain embodiments, the plurality of primary probes comprises optionally, a second primary probe, that interacts with a second gene segment. In certain embodiments, the plurality of primary probes comprises optionally, a third primary probe, that interacts with a third gene segment. In some embodiments, the first, second, and third primary probes are different from each other. In some embodiments, the method comprises imaging the cell population after the contacting step so that interaction of the probes with their gene segments are detected.

Using these methods, individual lymphocytes can be imaged in situ to determine tissue-wide clonality based on the rearrangement of T cell receptor gene segments. Post hoc bulk sequencing may allow for complementarity-determining sequences to be mapped back to individual lymphocyte clones in super resolution images. In addition to its clinical applications in histopathology, these methods may also be useful for the study of cellular and immunologic processes related to lymphoproliferative and autoimmune disorders.

### BRIEF DESCRIPTIONS OF THE DRAWINGS

FIG. 1. Spatial assessment of lymphocyte clonality by TCR seqFISH. FIG. 1A) TCR-clonotype specific probe design for gene segment identification in single transcripts using smFISH. Primary probes (black) were designed to target the variable (V), junction (J), and constant (C) regions of the JM22 TRB mRNA transcripts. Probes targeting the same gene segment were hybridized using readouts of one color, using colocalization of all 3 colors for identification of intact TCR transcripts by smFISH. FIG. 1B) Representative multicolor smFISH image of a Jurkat JM22 T cell with multiple clonotype-specific TRB transcripts (TRBV19, TRBJ2-7, TRBC2). Left: Single-channel images showing readout signal from JM22 TRB gene segments and nuclear staining (DAPI). Right: Composite multicolor image of Jurkat JM22. White dots represent transcripts colocalized in all 3 channels with probes targeting distinct TCR gene segments. The image represents a single z plane taken at the approximately widest point of the cell. Scale bar, 1.1 μm. FIG. 1C) Representative multicolor smFISH image of a Jurkat E6.1 cell used as a negative control because it lacks the JM22-specific TRB rearrangement. Left: Single-channel images showing readout signal from JM22 TRB gene segments and nuclear staining (DAPI). Right: Composite multicolor image of Jurkat E6.1. Dots do not colocalize between the channels, correctly identifying that Jurkat E6.1 lacks the JM22 TRB construct. The image represents a single z plane taken at the approximately widest point of the cell. Scale bar, 1.1 μm. FIG. 1D) HCR amplifies the signal from single primary probes hybridized to the TCR J segment in individual transcripts. Left: Representative image of Jurkat JM22 with smFISH imaging of TRBJ2-7 with calculated signal-to-noise ratio. Right: Representative image of Jurkat JM22 with imaging of TRBJ2-7 amplified by HCR with calculated signal-to-noise ratio. Note that smFISH for TRBJ2-7 was read out in the 647 nm channel while the HCR amplified signal was read out in the 561 nm channel. S/N Ratio, signal-to-noise ratio. Scale bar, 1.1 μm.

FIG. 2. Spatial assessment of lymphocyte clonality by TCR seqFISH. FIG. 2A) Diagram of single-cell spatial profiling of TCRs using seqFISH. Top: The sample undergoes multiple barcoding rounds, each consisting of multiple imaging rounds. The identity of individual transcripts is determined by the sequential barcode of individual points. Bottom: Diagram of a multicellular, spatially-organized tissue (skin) in which TCR seqFISH may be useful in characterizing T cell infiltrates. Right: Example gene barcodes organized by encoding channel. FIG. 2B) Representative pseudocolor image of a Jurkat JM22 T cell with colocalization for the 3-pseudocolor barcode for JM22 TRBV19. Left: Images of the hybridization rounds and channels corresponding to the barcode for TRBV19 and nuclear stain (DAPI). Right: Merged pseudocolor image showing colocalization for TRBV19 in Jurkat JM22 T cells. Pseudocolor images have been pre-processed to remove background noise.

FIG. 3. Demonstration of TCR seqFISH for single-cell evaluation of T cell polyclonality in whole tissue sections. FIG. 3A) Single hybridization and imaging round for TCR seqFISH on a 3×3 field-of-view of a germinal center and surrounding red pulp in a section of fresh frozen human spleen. The image is zoomed into a region at the border between red pulp and the germinal center. Dots from encoding channels are shown in cyan, magenta, and yellow. Nuclear staining (DAPI) is shown in white. Background 488 nm autofluorescence is included in red to show the locations of highly autofluorescent erythrocytes found in abundance in the splenic red pulp. FIG. 3B) Heatmap of cell-by-cell decoded TRBV genes from human spleen. Rows and columns represent V genes and single cells, respectively. Rows and columns have been hierarchically clustered as shown in the dendrograms. Shading represents total counts per cell.

FIG. 4. Analysis of shared V and J TCR gene segment usage among different T cell clones in a polyclonal human skin sample. Single-cell sequencing data of T cell receptors in suction blister fluid from a lesion of a patient with vitiligo. Left: Chord diagrams of V and J TCR gene segment usage of individual T cell clones for TRA and TRB. Each line connecting a V and J segment corresponds to a unique clonotype as defined by its CDR3 sequence. Chord width indicates multiple clones using the same V and J pair, but which can still differ in CDR3 sequence by differences in junctional indels or D gene segments. Right: Empirical cumulative density function of clones and the number that share a single V and J gene segment pairing for TRA and TRB. The majority of clones from this clonotype-enriched dataset are unique in their V and J pairing, supporting the feasibility of using post hoc bulk sequencing for mapping CDR3 sequence data to individual T cells in situ using TCR seqFISH.

FIG. 5. Programmable amplification for multiplexing TCR seqFISH. FIG. 5A) TCR-specific probe design for gene segment identification in single transcripts. Top: Probes were designed to target the variable (V), junction (J), and constant (C) regions of TCR mRNA transcripts. Middle: Iterative rounds of amplification allow for controlled amplification of a library of amplifiers corresponding to pseudocolors in TCR gene-encoding barcodes. Bottom: Fluorescently conjugated readout probes are sequentially hybridized to read out the set of amplifiers at each point, which can be decoded back to provide the gene segment identities of single transcripts.

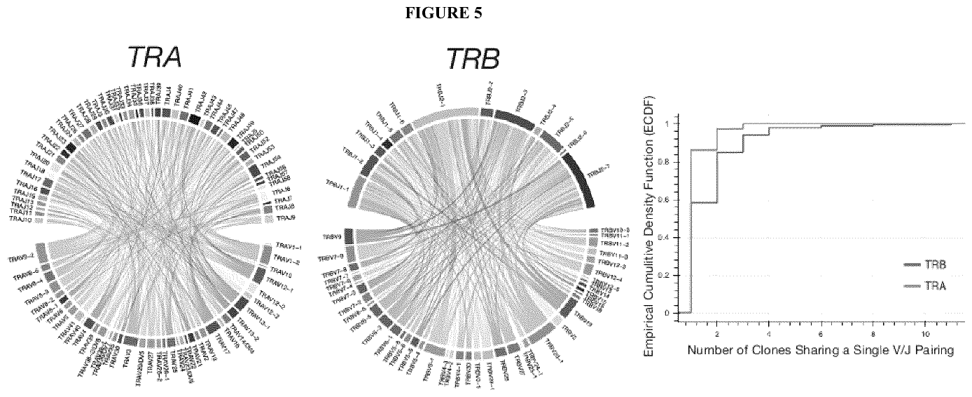

FIG. 6 Confocal microscopy images after amplification. From left to right, the two panels correspond to the 561 nm and 488 nm channels. From the top row to the bottom row, the three panels correspond to TRBC2, TRBV19, and TRBJ7-2. As illustrated, the amplification procedure yields clear fluorescent signals with excellent colocalization across channels.

FIG. 7 Schematics for spatial TCR mapping in single cells. Top, TCR mRNAs are hybridized by padlock probes targeting the V, J, and constant regions. TRB is shown as an example. SeqFISH is used to readout the identity of the V and J variants within each cell (middle panel, triple color stacks correspond to each decoded mRNA in cells). Consensus calling within each cell determines the TCR sequence within that cell, because TCR sequences are identical with a cell, and can mitigate dropouts in detection. Bottom panel, sequencing of the same tissue section or adjacent sections can identify the unique CDR3 region along with the corresponding V and J regions. Matching the V and J regions in the seqFISH data and the sequencing experiment allows mapping of the CDR3 sequence onto the TCR seqFISH images. Note, constant regions are identical in all sequences and seqFISH experiments.

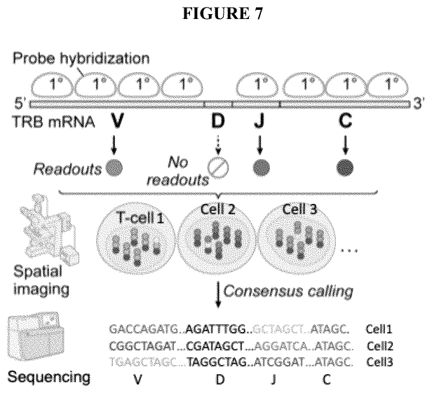

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### Definitions

Unless otherwise noted, terms are to be understood according to conventional usage by those of ordinary skill in the relevant art.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “oligonucleotide” refers to a polymer or oligomer of nucleotide monomers, containing any combination of nucleobases, modified nucleobases, sugars, modified sugars, phosphate bridges, or modified bridges. Oligonucleotides can be of various lengths. In particular embodiments, oligonucleotides can range from about 2 to about 1000 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length.

As used herein, the term “probe” or “probes” refers to any molecules, synthetic or naturally occurring, that can attach themselves directly or indirectly to a molecular target (e.g., an mRNA sample, DNA molecules, protein molecules, RNA and DNA isoform molecules, single nucleotide polymorphism molecules, and etc.). For example, a probe can include a nucleic acid molecule, an oligonucleotide, a protein (e.g., an antibody or an antigen binding sequence), or combinations thereof. For example, a protein probe may be connected with one or more nucleic acid molecules to for a probe that is a chimera. As disclosed herein, in some embodiments, a probe itself can produce a detectable signal. In some embodiments, a probe is connected, directly or indirectly via an intermediate molecule, with a signal moiety (e.g., a dye or fluorophore) that can produce a detectable signal.

As used herein, the term “binding sites” refer to a portion of a probe where other molecules may bind to the probe. In certain embodiments, the binding sites of a probe bind to another molecule through a non-covalent interaction.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to nucleic acid that has been extracted from the cell.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

As disclosed herein, the term “label” generally refers to a molecule that can recognize and bind to specific target sites within a molecular target in a cell. For example, a label can comprise an oligonucleotide that can bind to a molecular target in a cell. The oligonucleotide can be linked to a moiety that has affinity for the molecular target. The oligonucleotide can be linked to a first moiety that is capable of covalently linking to the molecular target. In certain embodiments, the molecular target comprises a second moiety capable of forming the covalent linkage with the label. In particular embodiments, a label comprises a nucleic acid sequence that is capable of providing identification of the cell which comprises or comprised the molecular target. In certain embodiments, a plurality of cells is labelled, wherein each cell of the plurality has a unique label relative to the other labelled cells.

As disclosed herein, the term “barcode” generally refers to a nucleotide sequence of a label produced by methods described herein. The barcode sequence typically is of a sufficient length and uniqueness to identify a single cell that comprises a molecular target.

As used herein the term “photoreactive group” refers to a chemically inert compound or molecule that becomes reactive when exposed to ultraviolet or visible light, that allows the formation of a covalent bond between two molecules.

As used herein, the term “complementary sequence” refers to a region of one nucleotide sequence that can base pair with another nucleotide sequence. In some embodiments, the complementary sequence can base pair with 60%, 65%, 70%, 75%, 80%, 85%, 90%, 91%, 92%, 93%, 94%, 95%, 96%, 97%, 98%, 99%, or 100% of another nucleotide sequence. In certain embodiments, the complementary sequence is between 5-1000 nucleotides long.

As used herein, the terms “binding”, “interacting”, or “interaction” refer to the association of two or more molecules. In some embodiments, “binding” or “interactions” are the result of electrostatic forces, hydrogen bonding, der Waals forces, salt bridges, and the hydrophobic effect. In certain embodiments, “interacting” or “interaction” refers to the interaction between two or more molecules. In certain embodiments, “interacting” or “interaction” refers to the hybridization of two nucleic acids. In certain embodiments, the interaction may be indirect, wherein one molecule interacts with another molecule by way of one or more intermediate molecules. In certain embodiments, the interaction of two or more molecules refers to the cross-linking of the molecules.

As used herein, the term “one or more” generally refers to a number range in a set with at least one object in the set. In certain embodiments, the term “one or more” refers to 1, 2, 3, 4, 5, 6, 7, 8, 9, 10, 11, 12, 13, 14, 15, 16, 17. 18, 19, 20, 25, 30, 35, 40, 45, 50, 60, 70, 80, 90, 100, 150, 200, 250, 300, 350, 400, 450, 500, 600, 700, 800, 900, or 1,000,000,000 objects in a set. In certain embodiments, the term “one or more” refers to a range between 1 to 1000 objects in a set. In certain embodiments, the term “one or more” refers to a range between 1 to 10,000 objects in a set. In certain embodiments, the term “one or more” refers to a range between 1 to 100,000 objects in a set. In certain embodiments, the term “one or more” refers to a range between 1 to 1,000,000 objects in a set. In certain embodiments, the term “one or more” refers to a range between 1 to 10,000,000 objects in a set. In certain embodiments, the term “one or more” refers to a range between 1 to 100,000,000 objects in a set. In certain embodiments, the term “one or more” refers to a range between 1 to 500,000,000 objects in a set. For instance, using “one or more” oligonucleotides may refer to using 1 to 1000 oligonucleotides, or any number or range in between.

As used herein, the term “two or more” generally refers to a number range in a set with at least one object in the set. In certain embodiments, the term “two or more” refers to 2, 3, 4, 5, 6, 7, 8, 9, 10, 11, 12, 13, 14, 15, 16, 17. 18, 19, 20, 25, 30, 35, 40, 45, 50, 60, 70, 80, 90, 100, 150, 200, 250, 300, 350, 400, 450, 500, 600, 700, 800, 900, or 1,000,000,000 objects in a set. In certain embodiments, the term “two or more” refers to a range between 2 to 1000 objects in a set. In certain embodiments, the term “two or more” refers to a range between 2 to 100,000 objects in a set. In certain embodiments, the term “two or more” refers to a range between 2 to 1,000,000 objects in a set. In certain embodiments, the term “two or more” refers to a range between 2 to 10,000,000 objects in a set. In certain embodiments, the term “two or more” refers to a range between 2 to 100,000,000 objects in a set. In certain embodiments, the term “two or more” refers to a range between 2 to 500,000,000 objects in a set. For instance, using “two or more” oligonucleotides may refer to using 2 to 1000 oligonucleotides, or any number or range in between.

As used herein, the term “part of” in the phrase refers to 1%, 5%, 10%, 15%, 20%, 25%, 30%, 35%, 40%, 45%, 50%, 55%, 60%, 65%, 70%, 75%, 80%, 85%, 90%, 95%, or 99% of a gene and/or gene segment. For instance, the phrase “[the]gene segment encodes part of a T cell receptor gene” refers to a gene segment that encodes 1%, 5%, 10%, 15%, 20%, 25%, 30%, 35%, 40%, 45%, 50%, 55%, 60%, 65%, 70%, 75%, 80%, 85%, 90%, 95%, or 99% of a gene and/or gene segment.

As used herein, the term “establish lineage information” refers to the process of determining how progeny cells are related to parental cells. This can be done by analyzing change by analyzing how mutations, deletions, insertions, duplications of gene segments, rearrangements of gene segments, or any combinations thereof occur between generations of cells propagated.

As used herein, the term “indicates changes” refers to mutations, deletions, insertions, duplications of gene segments, rearrangements of gene segments, or any combination thereof.

As used herein, the term “in proximity” refers to the nucleotide distance on either side of a gene segment. In some embodiments, the distance on either side is 5, 10, 100, 200, 300, 400, 500, 600, 700, 800, 900, 1000, 2000, 3000, 4000, 5000, 6000, 7000, 8000, 9000, 10000, 12000, 14000, 16000, 18000, 20000, 25000, or 30000 nucleotides.

As used herein, the term “amplification” refers to the exponential or geometric growth in the number of copies of a molecule.

As used herein, the term “targets” or “molecular targets” refers to molecules that are selected from proteins, modified proteins, transcripts, gene segments, RNA, DNA loci, exogenous proteins, exogenous nucleic acids, hormones, carbohydrates, small molecules, biologically active molecules, and combinations thereof.

As used herein, the term “gene segment” refers to a target or molecular target.

As used herein, the term “complementary sequence” refers to a region of one nucleotide sequence that can base pair with another nucleotide sequence. In some embodiments, the complementary sequence can base pair with 60%, 65%, 70%, 75%, 80%, 85%, 90%, 91%, 92%, 93%, 94%, 95%, 96%, 97%, 98%, 99%, or 100% of another nucleotide sequence. In certain embodiments, the complementary sequence is between 5-1000 nucleotides long.

As used herein, the term “cellular components” are selected from oligonucleotides, DNAs, RNAs, small molecules, glycans, peptides, proteins, antibodies, antibodies conjugated to oligonucleotides, organelles, subcellular compartments, and combinations thereof.

As used herein, the term “pairings” as used in V, D, and J pairings refers to the pairs formed through V(D)J recombination in developing lymphocytes that assembles unique antigen receptors by pairing Variable (V), Diversity (D), and Joining (J) gene segments, or combinations thereof.

As used herein, the term “changes” as used in changes to the variable (V), joining (J), or a diversity (D) segments in the cells of the cell population refers to the recombination of V, D, or J segments within the genome or transcripts of the cells of the cell population.

### EMBODIMENTS

In some embodiments, a method is described comprising contacting a cell population with a plurality of primary probes. In certain embodiments, the plurality of primary probes comprises a first primary probe that interacts with a first gene segment. In certain embodiments, the plurality of primary probes comprises optionally, a second primary probe, that interacts with a second gene segment. In certain embodiments, the plurality of primary probes comprises optionally, a third primary probe, that interacts with a third gene segment. In some embodiments, the first, second, and third primary probes are different from each other. In some embodiments, the method comprises imaging the cell population after the contacting step so that interaction of the probes with their gene segments are detected.

In some embodiments, the plurality of primary probes in any of the previous embodiments comprises the first primary probe and the second primary probe.

In some embodiments, the plurality of primary probes in any of the previous embodiments comprises the first primary probe, the second primary probe, and the third primary probe.

In some embodiments, the method of any of the previous embodiments further comprises optionally, contacting a plurality of secondary probes to the primary probes before imaging the cell population after the contacting step so that interaction of the probes with their gene segments are detected.

In some embodiments, the method of any of the previous embodiments further comprises optionally, contacting a plurality of tertiary probes to the secondary probes before imaging the cell population after the contacting step so that interaction of the probes with their gene segments are detected.

In some embodiments, the method of any of the previous embodiments further comprises optionally, contacting a plurality of quaternary probes to the tertiary probes before imaging the cell population after the contacting step so that interaction of the probes with their gene segments are detected.

In some embodiments, the method of any of the previous embodiments further comprises contacting the primary probes, secondary probes, tertiary probes, and/or quaternary probes with one or more readout probes before imaging the cell population after the contacting step so that interaction of the probes with their gene segments are detected.

In some embodiments, the method of any of the previous embodiments further comprises optionally, stabilizing the primary, secondary, tertiary, and/or quaternary probes during or after each contacting step.

In some embodiments, the method of any of the previous embodiments further comprises optionally, washing the cell population to remove the primary probes, secondary probes, tertiary probes, quaternary probes, and/or readout probes. In some embodiments, the method of any of the previous embodiments, further comprises sequencing nucleic acid sequences in proximity to the gene segments. In some embodiments, the method of any of the previous embodiments, comprises correlating the nucleic acid sequences to detected gene segments in the cells of the cell population.

In some embodiments, the method of any of the previous embodiments further comprises optionally, sectioning the tissue into one or more tissue sections before contacting the cell population with a plurality of primary probes. In some embodiments, the method of any of the previous embodiments comprises forming a map of one of the tissue sections, wherein the map correlates the nucleic acid read sequences to their detected gene segments in the cells of the tissue section. In some embodiments, the method of any of the previous embodiments comprises assembling a global map comprising two or more maps of tissue sections.

### Samples and Gene Segment

In some embodiments, the method comprises analyzing samples, wherein the samples comprise cell populations. In certain embodiments, the samples are the cells of a cell population.

In some embodiments, the cell population comprises lymphocytes. In certain embodiments, the lymphocytes are T-lymphocytes and B-lymphocytes. In certain embodiments, the lymphocytes are T-lymphocytes. In certain embodiments, the lymphocytes are B-lymphocytes.

In some embodiments, the cell population is a tissue sample. In certain embodiments, the method comprises sectioning the tissue sample into one or more sections of tissue. In certain embodiments, the cell population is a section of the one or more sections of tissue.

In some embodiments, the first, second, and/or third gene segments are in the cells of the cell population. In some embodiments, the gene segments are in the genome of the cells of the cell population. In some embodiments, the gene segments are in one or more RNA transcripts of the cells of the cell population.

In certain embodiments, each gene segment encodes part of a T cell receptor gene or immunoglobulin (Ig) protein. In certain embodiments, the gene segments are variable (V) joining (J), and/or diversity (D) segments. In certain embodiments, the first gene segment is a variable (V) or joining segment (J). In certain embodiments, the second gene segment is a variable (V) or joining segment (J). In certain embodiments, the first primary probe interacts with the V segment, and wherein the second primary probe interacts with the J segment. In certain embodiments, the first primary probe interacts with the J segment, and wherein the second primary probe interacts with the V segment.

### Primary, Secondary, Tertiary, and Quaternary Probes

In some embodiments, the method comprises contacting primary probes, secondary probes, tertiary probes, and/or quaternary probes to the cells of the cell population.

In some embodiments, a method is described comprising contacting a cell population with a plurality of primary probes. In some embodiments, the first, second, and/or third primary probes comprise one or more secondary probe binding sites and/or one or more readout probe binding sites. In some embodiments, the first, second, and/or third primary probes comprise a nucleic acid sequence complementary to a gene segment. In some embodiments, the nucleic acid sequence is 60%, 65%, 70%, 75%, 80%, 85%, 90%, 91%, 92%, 93%, 94%, 95%, 96%, 97%, 98%, 99%, or 100% complementary to the gene segment. In some embodiments, the complementary sequence is between 5-1000 nucleotides long.

In some embodiments, the primary probe of any of the preceding embodiments comprises oligonucleotides that are at least 5 nucleotides in length. In some embodiments, the primary probe of any of the preceding embodiments comprises oligonucleotides that are at least 6 nucleotides in length. In some embodiments, the primary probe of any of the preceding embodiments comprises oligonucleotides that are at least 7 nucleotides in length. In some embodiments, the primary probe of any of the preceding embodiments comprises oligonucleotides that are at least 8 nucleotides in length. In some embodiments, the primary probe of any of the preceding embodiments comprises oligonucleotides that are at least 9 nucleotides in length. In some embodiments, the primary probe of any of the preceding embodiments comprises oligonucleotides that are at least 10 nucleotides in length. In some embodiments, the primary probe of any of the preceding embodiments comprises oligonucleotides that are at least 11 nucleotides in length. In some embodiments, the primary probe of any of the preceding embodiments comprises oligonucleotides that are at least 12 nucleotides in length. In some embodiments, the primary probe of any of the preceding embodiments comprises oligonucleotides that are at least 13 nucleotides in length. In some embodiments, the primary probe of any of the preceding embodiments comprises oligonucleotides that are at least 14 nucleotides in length. In some embodiments, the primary probe of any of the preceding embodiments comprises oligonucleotides that are at least 15 nucleotides in length. In some embodiments, the primary probe of any of the preceding embodiments comprises oligonucleotides that are at least 16 nucleotides in length. In some embodiments, the primary probe of any of the preceding embodiments comprises oligonucleotides that are at least 17 nucleotides in length. In some embodiments, the primary probe of any of the preceding embodiments comprises oligonucleotides that are at least 18 nucleotides in length. In some embodiments, the primary probe of any of the preceding embodiments comprises oligonucleotides that are at least 19 nucleotides in length. In some embodiments, the primary probe of any of the preceding embodiments comprises oligonucleotides that are at least 20 nucleotides in length. In some embodiments, the primary probe of any of the preceding embodiments comprises oligonucleotides that are at least 21 nucleotides in length. In some embodiments, the primary probe of any of the preceding embodiments comprises oligonucleotides that are less than 30, 50, 100, 200, 250, 500, 750, or 1000 nucleotides in length.

In some embodiments, the primary, secondary, tertiary, or quaternary probe comprises at least one readout probe binding site. In certain embodiments, in any of the previous embodiments, the primary, secondary, tertiary, or quaternary probe comprises at least two readout probe binding sites. In some embodiments, in any of the previous embodiments, the primary, secondary, tertiary, or quaternary probe comprises at least three readout probe binding sites. In some embodiments, in any of the previous embodiments, the primary, secondary, tertiary, or quaternary probe comprises at least four readout probe binding sites. In some embodiments, in any of the previous embodiments, the primary, secondary, tertiary, or quaternary probe comprises at least five readout probe binding sites. In some embodiments, in any of the previous embodiments, the primary, secondary, tertiary, or quaternary probe comprises at least six readout probe binding sites. In some embodiments, in any of the previous embodiments, the primary, secondary, tertiary, or quaternary probe comprises at least seven readout probe binding sites. In some embodiments, in any of the previous embodiments, the primary, secondary, tertiary, or quaternary probe comprises at least eight readout probe binding sites. In some embodiments, in any of the previous embodiments, the primary, secondary, tertiary, or quaternary probe comprises at least nine readout probe binding sites. In some embodiments, in any of the previous embodiments, the primary, secondary, tertiary, or quaternary probe comprises at least 10 readout probe binding sites.

In some embodiments, each secondary probe comprises one or more tertiary probe binding sites and/or one or more readout probe binding sites, and wherein each secondary probe in the plurality of the secondary probes interacts with either the first primary probe, second primary probe, and/or third primary probe.

In some embodiments, the secondary probe of any of the preceding embodiments comprises oligonucleotides that are at least 5 nucleotides in length. In some embodiments, the secondary probe of any of the preceding embodiments comprises oligonucleotides that are at least 6 nucleotides in length. In some embodiments, the secondary probe of any of the preceding embodiments comprises oligonucleotides that are at least 7 nucleotides in length. In some embodiments, the secondary probe of any of the preceding embodiments comprises oligonucleotides that are at least 8 nucleotides in length. In some embodiments, the secondary probe of any of the preceding embodiments comprises oligonucleotides that are at least 9 nucleotides in length. In some embodiments, the secondary probe of any of the preceding embodiments comprises oligonucleotides that are at least 10 nucleotides in length. In some embodiments, the secondary probe of any of the preceding embodiments comprises oligonucleotides that are at least 11 nucleotides in length. In some embodiments, the secondary probe of any of the preceding embodiments comprises oligonucleotides that are at least 12 nucleotides in length. In some embodiments, the secondary probe of any of the preceding embodiments comprises oligonucleotides that are at least 13 nucleotides in length. In some embodiments, the secondary probe of any of the preceding embodiments comprises oligonucleotides that are at least 14 nucleotides in length. In some embodiments, the secondary probe of any of the preceding embodiments comprises oligonucleotides that are at least 15 nucleotides in length. In some embodiments, the secondary probe of any of the preceding embodiments comprises oligonucleotides that are at least 16 nucleotides in length. In some embodiments, the secondary probe of any of the preceding embodiments comprises oligonucleotides that are at least 17 nucleotides in length. In some embodiments, the secondary probe of any of the preceding embodiments comprises oligonucleotides that are at least 18 nucleotides in length. In some embodiments, the secondary probe of any of the preceding embodiments comprises oligonucleotides that are at least 19 nucleotides in length. In some embodiments, the secondary probe of any of the preceding embodiments comprises oligonucleotides that are at least 20 nucleotides in length. In some embodiments, the secondary probe of any of the preceding embodiments comprises oligonucleotides that are at least 21 nucleotides in length. In some embodiments, the secondary probe of any of the preceding embodiments comprises oligonucleotides that are less than 30, 50, 100, 200, 250, 500, 750, or 1000 nucleotides in length.

In some embodiments, each tertiary probe comprises one or more quaternary probe binding sites and/or one or more readout probe binding sites, and wherein each tertiary probe in the plurality of the tertiary probes interacts with one of the secondary probes in the plurality of secondary probes.

In some embodiments, the tertiary probe of any of the preceding embodiments comprises oligonucleotides that are at least 5 nucleotides in length. In some embodiments, the tertiary probe of any of the preceding embodiments comprises oligonucleotides that are at least 6 nucleotides in length. In some embodiments, the tertiary probe of any of the preceding embodiments comprises oligonucleotides that are at least 7 nucleotides in length. In some embodiments, the tertiary probe of any of the preceding embodiments comprises oligonucleotides that are at least 8 nucleotides in length. In some embodiments, the tertiary probe of any of the preceding embodiments comprises oligonucleotides that are at least 9 nucleotides in length. In some embodiments, the tertiary probe of any of the preceding embodiments comprises oligonucleotides that are at least 10 nucleotides in length. In some embodiments, the tertiary probe of any of the preceding embodiments comprises oligonucleotides that are at least 10 nucleotides in length In some embodiments, the tertiary probe of any of the preceding embodiments comprises oligonucleotides that are at least 11 nucleotides in length. In some embodiments, the tertiary probe of any of the preceding embodiments comprises oligonucleotides that are at least 12 nucleotides in length. In some embodiments, the tertiary probe of any of the preceding embodiments comprises oligonucleotides that are at least 13 nucleotides in length. In some embodiments, the tertiary probe of any of the preceding embodiments comprises oligonucleotides that are at least 14 nucleotides in length. In some embodiments, the tertiary probe of any of the preceding embodiments comprises oligonucleotides that are at least 15 nucleotides in length. In some embodiments, the tertiary probe of any of the preceding embodiments comprises oligonucleotides that are at least 16 nucleotides in length. In some embodiments, the tertiary probe of any of the preceding embodiments comprises oligonucleotides that are at least 17 nucleotides in length. In some embodiments, the tertiary probe of any of the preceding embodiments comprises oligonucleotides that are at least 18 nucleotides in length. In some embodiments, the tertiary probe of any of the preceding embodiments comprises oligonucleotides that are at least 19 nucleotides in length. In some embodiments, the tertiary probe of any of the preceding embodiments comprises oligonucleotides that are at least 20 nucleotides in length. In some embodiments, the tertiary probe of any of the preceding embodiments comprises oligonucleotides that are at least 21 nucleotides in length. In some embodiments, the tertiary probe of any of the preceding embodiments comprises oligonucleotides that are less than 30, 50, 100, 200, 250, 500, 750, or 1000 nucleotides in length.

In some embodiments, each quaternary probe comprises one or more readout probe binding sites, and wherein each quaternary probe in the plurality of the quaternary probes interacts with one of the tertiary probes in the plurality of tertiary probes.

In some embodiments, the quaternary probe of any of the preceding embodiments comprises oligonucleotides that are at least 5 nucleotides in length. In some embodiments, the quaternary probe of any of the preceding embodiments comprises oligonucleotides that are at least 6 nucleotides in length. In some embodiments, the quaternary probe of any of the preceding embodiments comprises oligonucleotides that are at least 7 nucleotides in length. In some embodiments, the quaternary probe of any of the preceding embodiments comprises oligonucleotides that are at least 8 nucleotides in length. In some embodiments, the quaternary probe of any of the preceding embodiments comprises oligonucleotides that are at least 9 nucleotides in length. In some embodiments, the quaternary probe of any of the preceding embodiments comprises oligonucleotides that are at least 10 nucleotides in length. In some embodiments, the quaternary probe of any of the preceding embodiments comprises oligonucleotides that are at least 10 nucleotides in length In some embodiments, the quaternary probe of any of the preceding embodiments comprises oligonucleotides that are at least 11 nucleotides in length. In some embodiments, the quaternary probe of any of the preceding embodiments comprises oligonucleotides that are at least 12 nucleotides in length. In some embodiments, the quaternary probe of any of the preceding embodiments comprises oligonucleotides that are at least 13 nucleotides in length. In some embodiments, the quaternary probe of any of the preceding embodiments comprises oligonucleotides that are at least 14 nucleotides in length. In some embodiments, the quaternary probe of any of the preceding embodiments comprises oligonucleotides that are at least 15 nucleotides in length. In some embodiments, the quaternary probe of any of the preceding embodiments comprises oligonucleotides that are at least 16 nucleotides in length. In some embodiments, the quaternary probe of any of the preceding embodiments comprises oligonucleotides that are at least 17 nucleotides in length. In some embodiments, the quaternary probe of any of the preceding embodiments comprises oligonucleotides that are at least 18 nucleotides in length. In some embodiments, the quaternary probe of any of the preceding embodiments comprises oligonucleotides that are at least 19 nucleotides in length. In some embodiments, the quaternary probe of any of the preceding embodiments comprises oligonucleotides that are at least 20 nucleotides in length. In some embodiments, the quaternary probe of any of the preceding embodiments comprises oligonucleotides that are at least 21 nucleotides in length. In some embodiments, the quaternary probe of any of the preceding embodiments comprises oligonucleotides that are less than 30, 50, 100, 200, 250, 500, 750, or 1000 nucleotides in length.

In some embodiments, the secondary probe complements the secondary probe binding site on the primary probe. In some embodiments, the tertiary probe complements the secondary probe binding site on the secondary probe. In some embodiments, the quaternary probe complements the tertiary probe binding site on the tertiary probe. In some embodiments, the probe complements comprise a sequence complementarity that is at least 60%, 65%, 70%, 75%, 80%, 85%, 90%, 91%, 92%, 93%, 94%, 95%, 96%, 97%, 98%, 99%, or 100%.

In some embodiments, the length of the primary, secondary, tertiary, and quaternary probe binding sites range from 5-100 nucleotides. In some embodiments, the length of the primary, secondary, tertiary, and quaternary probe binding sites range from 5-10 nucleotides. In some embodiments, the length of the primary, secondary, tertiary, and quaternary probe binding sites range from 5-20 nucleotides. In some embodiments, the length of the primary, secondary, tertiary, and quaternary probe binding sites range from 5-30 nucleotides. In some embodiments, the length of the primary, secondary, tertiary, and quaternary probe binding sites range from 5-40 nucleotides. In some embodiments, the length of the primary, secondary, tertiary, and quaternary probe binding sites range from 5-50 nucleotides. In some embodiments, the length of the primary, secondary, tertiary, and quaternary probe binding sites range from 5-60 nucleotides. In some embodiments, the length of the primary, secondary, tertiary, and quaternary probe binding sites range from 5-70 nucleotides. In some embodiments, the length of the primary, secondary, tertiary, and quaternary probe binding sites range from 5-80 nucleotides. In some embodiments, the length of the primary, secondary, tertiary, and quaternary probe binding sites range from 5-90 nucleotides.

### Readout Probe

In some embodiments, the one or more readout probes of any of the previous embodiments comprise an oligonucleotide or antibody with a detectable moiety.

In some embodiments, the one or more readout probes of any of the preceding embodiments comprise oligonucleotides with the same sequence.

In some embodiments, the one or more readout probes of any of the preceding embodiments comprise oligonucleotides with different sequences.

In some embodiments, the one or more readout probes of any of the preceding embodiments comprise oligonucleotides that are at least 17 nucleotides in length.

In some embodiments, the readout probe of any of the preceding embodiments comprises oligonucleotides that are at least 5 nucleotides in length. In some embodiments, the readout probe of any of the preceding embodiments comprises oligonucleotides that are at least 10 nucleotides in length. In some embodiments, the readout probe of any of the preceding embodiments comprises oligonucleotides that are at least 11 nucleotides in length. In some embodiments, the readout probe of any of the preceding embodiments comprises oligonucleotides that are at least 12 nucleotides in length. In some embodiments, the readout probe of any of the preceding embodiments comprises oligonucleotides that are at least 13 nucleotides in length. In some embodiments, the readout probe of any of the preceding embodiments comprises oligonucleotides that are at least 14 nucleotides in length. In some embodiments, the readout probe of any of the preceding embodiments comprises oligonucleotides that are at least 15 nucleotides in length. In some embodiments, the readout probe of any of the preceding embodiments comprises oligonucleotides that are at least 16 nucleotides in length. In some embodiments, the readout probe of any of the preceding embodiments comprises oligonucleotides that are at least 17 nucleotides in length. In some embodiments, the readout probe of any of the preceding embodiments comprises oligonucleotides that are at least 18 nucleotides in length. In some embodiments, the readout probe of any of the preceding embodiments comprises oligonucleotides that are at least 19 nucleotides in length. In some embodiments, the readout probe of any of the preceding embodiments comprises oligonucleotides that are at least 20 nucleotides in length. In some embodiments, the readout probe of any of the preceding embodiments comprises oligonucleotides that are at least 21 nucleotides in length. In some embodiments, the readout probe of any of the preceding embodiments comprises oligonucleotides that are less than 30, 50, 100, 200, 250, 500, 750, or 1000 nucleotides in length.

In some embodiments, the readout probe complements the readout probe binding site on the primary probe. In some embodiments, the readout probe complements the readout probe binding site on the secondary probe. In some embodiments, the readout probe complements the readout probe binding site on the tertiary probe. In some embodiments, the readout probe complements the readout probe binding site on the quaternary probe. In some embodiments, the readout probe complements to a splint sequence fragment. In some embodiments, the probe complements comprise a sequence complementarity that is at least 60%, 65%, 70%, 75%, 80%, 85%, 90%, 91%, 92%, 93%, 94%, 95%, 96%, 97%, 98%, 99%, or 100%.

In some embodiments, the length of the readout probe binding sites range from 5-100 nucleotides. In some embodiments, the length of the readout probe binding sites range from 5-10 nucleotides. In some embodiments, the length of the readout probe binding sites range from 5-20 nucleotides. In some embodiments, the length of the readout probe binding sites range from 5-30 nucleotides. In some embodiments, the length of the readout probe binding sites range from 5-40 nucleotides. In some embodiments, the length of the readout probe binding sites range from 5-50 nucleotides. In some embodiments, the length of the readout probe binding sites range from 5-60 nucleotides. In some embodiments, the length of the readout probe binding sites range from 5-70 nucleotides. In some embodiments, the length of the readout probe binding sites range from 5-80 nucleotides. In some embodiments, the length of the readout probe binding sites range from 5-90 nucleotides.

In some embodiments, the method comprises a readout probe hybridizing to one or more of the primary, secondary, tertiary, and/or quaternary probes.

In some embodiments, the method comprises barcoding the gene segments in the cells of the cell population. In some embodiments, the methods of any of preceding embodiments, further comprises repeating the contacting and imaging steps, each time with a new plurality of detectably labeled readout probes, wherein in each new plurality at least one readout probe for one target differs from at least one readout probe for the same target in a previous plurality, wherein they differ at least in their detectable moieties.

In some embodiments, the one or more readout probes are sequencing probes.

### Fluorophores

In some embodiments, the methods of any of the embodiments comprise a detectable moiety. In some embodiments, the methods of any one of the preceding embodiments comprises at least two different detectable moieties. In certain embodiments, the detectable moieties are the same.

In some embodiments, the detectable moiety is any fluorophore deemed suitable by those of skill in the arts.

In some embodiments, the detectable moieties include but are not limited to fluorescein, rhodamine, Alexa Fluors, DyLight fluors, ATTO Dyes, or any analogs or derivatives thereof. In certain embodiments, the detectable moieties include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some embodiments, the detectable moieties include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl) naphthacene; 9,10-Bis(phenylethynyl)anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenylethynyl)anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate) ruthenium(II), Texas Red, TSQ, Umbelliferone, or Yellow fluorescent protein.

In some embodiments, the detectable moieties include but are not limited to Alexa Fluor family of fluorescent dyes (Molecular Probes, Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. In some embodiments, sulfonation makes Alexa Fluor dyes negatively charged and hydrophilic. In some embodiments, Alexa Fluor dyes are more stable, brighter, and less pH-sensitive than common dyes (e.g. fluorescein, rhodamine) of comparable excitation and emission, and to some extent the newer cyanine series. Exemplary Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750.

In some embodiments, the detectable moieties comprise one or more of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, the detectable moieties comprises a nanomaterial. In some embodiments, the fluorophore is a nanoparticle. In some embodiments, the detectable moiety is or comprises a quantum dot. In some embodiments, the fluorophore is a quantum dot. In some embodiments, the detectable moiety comprises a quantum dot. In some embodiments, the detectable moiety is or comprises a gold nanoparticle. In some embodiments, the detectable moiety is a gold nanoparticle. In some embodiments, the detectable moiety comprises a gold nanoparticle.

### Removing Probes

In some embodiments, the method of any of the preceding embodiments, comprises washing the sample after each step. In certain embodiments, the sample is washed with a buffer that removes non-specific hybridization reactions. In certain embodiments, formamide is used in the wash step. In certain embodiments, the wash buffer is stringent. In certain embodiments, the wash buffer comprises 10% formamide, 2×SSC, and 0.1% triton X-100s.

In some embodiments, the method comprises a step of removing the one or more probes after one or more imaging steps. In some embodiments, the step of removing the probes comprises contacting the plurality of readout probes with an enzyme that digests the probes. In some embodiments, the step of removing comprises contacting the plurality of probes with a DNase, contacting the plurality of probes with an RNase, photobleaching, strand displacement, formamide wash, heat denaturation, chemical denaturation, cleavage, or combinations thereof. In some embodiments, the step of removing comprises photobleaching to remove the probes.

In some embodiments, the method further comprises comprising removing the readout probes after one or more imaging steps. In some embodiments, the method comprises the step of removing comprises contacting the plurality of readout probes with an enzyme that digests a readout probe. In some embodiments, the method comprises removing the readout probes by using stripping reagents, wash buffers, photobleaching, chemical bleaching, and any combinations thereof. In some embodiments, the method comprises contacting the plurality of readout probes with a DNase, contacting the plurality of probes with an RNase, photobleaching, strand displacement, formamide wash, heat denaturation, or combinations thereof. In some embodiments, the readout probes are removed by photobleaching.

In some embodiments, the method comprises clearing the sample. In some embodiments the sample is cleared by CLARITY. In some embodiments, the sample is cleared following hydrogel embedding.

Certain techniques for removing probes are known in the art. See, for example:
a. International PCT Patent Application No. PCT/US2014/036258, filed Apr. 30, 2014, and titled MULTIPLEX LABELING OF MOLECULES BY SEQUENTIAL HYBRIDIZATION BARCODING, the entire contents of which are herein incorporated by reference in its entirety for all purposes.
b. International PCT Patent Application No. PCT/US2018/064616, filed Dec. 7, 2018, and titled MULTIPLEX LABELING OF MOLECULES Publication Classification BY SEQUENTIAL HYBRIDIZATION BARCODING WITH RAPID SWITCHING AND REHYBRIDIZATION OF PROBES, the entire contents of which are herein incorporated by reference in its entirety for all purposes.
c. International PCT Patent Application No. PCT/US2017/044994, filed Aug. 1, 2017, and titled SEQUENTIAL PROBING OF MOLECULAR TARGETS BASED ON PSEUDO—COLOR BARCODES WITH EMBEDDED ERROR CORRECTION MECHANISM, the entire contents of which are herein incorporated by reference in its entirety for all purposes.
d. International PCT Patent Application No. PCT/US2022/024494, filed Apr. 12, 2022, and titled HIGH-RESOLUTION WHOLE GENOME IMAGING BY NUCLEIC ACID LOCUS AND BLOCK CODING, the entire contents of which are herein incorporated by reference in its entirety for all purposes.
e. International PCT Patent Application No. PCT/US2022/032736, filed Jun. 8, 2022, and titled RATIOMETRIC SYMBOLS AND SEQUENTIAL CODING FOR MULTIPLEXED FISH, the entire contents of which are herein incorporated by reference in its entirety for all purposes.
f. International PCT Patent Application No. PCT/US2022/053995, filed Dec. 23, 2022, and titled SUPPRESSION OF NON-SPECIFIC SIGNALS BY EXONUCLEASES IN FISH EXPERIMENT, the entire contents of which are herein incorporated by reference in its entirety for all purposes.
g. International PCT Patent Application No. PCT/US2022/051737, filed Dec. 2, 2022, and titled METHOD OF MAPPING SPATIAL DISTRIBUTIONS OF CELLULAR COMPONENTS, the entire contents of which are herein incorporated by reference in its entirety for all purposes.
h. International PCT Patent Application No. PCT/US2022/017757, filed Feb. 24, 2022, and titled MULTIPLEXING OF EXPERIMENTAL CONDITIONS AND SAMPLES IN SPATIAL GENOMICS, the entire contents of which are herein incorporated by reference in its entirety for all purposes.
i. International PCT Patent Application No. PCT/US2025/015646, filed Feb. 12, 2025, and titled SUPER-RESOLVED OBJECT DETECTION WITH SPATIAL GENOMICS, the entire contents of which are herein incorporated by reference in its entirety for all purposes.

### Stabilization

In some embodiments, the methods of any of the previous embodiments further comprise stabilizing the primary probes, secondary probes, tertiary probes, and/or quaternary probes during or after each contacting step. In certain embodiments, the primary probes, secondary probes, tertiary probes, and/or quaternary probes are stabilized by ligating the probes to themselves or to cellular components. In certain embodiments, the primary probes, secondary probes, tertiary probes, and/or quaternary probes are stabilized by crosslinking, such as photo-crosslinking or chemical crosslinking.

### Analysis of Samples

In some embodiments, the method comprises analyzing nucleic acids by barcoding, sequencing, and histological methods.

In some embodiments, the cell population is sequenced. In some embodiments, the lymphocyte population is genomically sequenced. In some embodiments, a cell-sorted lymphocyte population is genomically sequenced. In certain embodiments, the cell-sorted lymphocyte population is sorted by fluorescence-activated cell sorting (FACS).

In some embodiments, the cell population is sequenced. In certain embodiments, the sequencing is by sequencing-by-ligation methods. In certain embodiments, the sequencing is by sequencing-by synthesis methods. In certain embodiments, the sequencing is by long-read sequencing methods.

In some embodiments, the sequencing is performed by amplifying nucleic acids by polymerase chain reaction to produce PCR amplicons. In certain embodiments, the PCR amplicons are sequenced.

In some embodiments, the method comprises sequencing the sequencing probes by sequencing-by-ligation. In some embodiments, the method comprises sequencing the sequencing probes by sequencing-by-synthesis. In some embodiments, the method comprises sequencing the sequencing probes by sequencing-by-long-read sequencing.

In some embodiments, the method further comprises a histological analysis of the lymphocyte population.

In some embodiments, the method of any of the previous embodiments comprises further comprising sorting the lymphocyte population to separate lymphocytes with different barcodes. In certain embodiments, the sorting method comprises using flow cytometry to sort cells. In certain embodiments, the method comprises genomically sequencing the sorted lymphocyte population. In certain embodiments, the genomic sequencing sequences DNA, mRNA, or any combination thereof.

In some embodiments, the methods of any of the previous embodiments further comprise determining a structure for a T cell receptor or an immunoglobulin protein.

In some embodiments, the method of any of the previous embodiments further comprises establishing the lineage information for the cells in the cell population of the barcoded cells. In certain embodiments, the lineage information indicates changes to the variable (V), joining (J), or diversity (D) segments to each cell in the cell population.

In some embodiments, the method comprises localizing the gene segments of T cell receptors or immunoglobulins in a sample. In certain embodiments, the gene segments are V, J, and/or D segments pairings detected by hybridization of probes and imaging. In certain embodiments, the gene segments are V and J segments pairings detected by hybridization of probes and imaging. In certain embodiments, the gene segments are V and/or J segments detected by hybridization of probes and imaging.

In some embodiments, the method comprises barcoding the gene segments of T cell receptors or immunoglobulins in a sample. In certain embodiments, the gene segments are V, J, and/or D segments pairings detected by hybridization of probes and imaging. In certain embodiments, the gene segments are V and J segments pairings detected by hybridization of probes and imaging. In certain embodiments, the gene segments are V and/or J segments detected by hybridization of probes and imaging.

In some embodiments, the method comprises correlating nucleotide mutations determined by sequencing with changes to the variable (V), joining (J), or diversity (D) segments detected in the cells in the cell population.

### Imaging the Sample

In some embodiments, the method comprises imaging the cell population after the contacting step so that interaction of the probes with their gene segments are detected. As understood by a person having ordinary skill in the art, different technologies can be used for the imaging steps.

In some embodiments, the imaging methods comprise but are not limited to epi-fluorescence microscopy, confocal microscopy, the different types of super-resolution microscopy (PALM/STORM, SSIM/GSD/STED), and light sheet microscopy (SPIM and etc).

In some embodiments, the imaging methods comprise exemplary super resolution technologies include, but are not limited to I⁵M and 4Pi-microscopy, Stimulated Emission Depletion microscopy (STEDM), Ground State Depletion microscopy (GSDM), Spatially Structured Illumination microscopy (SSIM), Photo-Activated Localization Microscopy (PALM), Reversible Saturable Optically Linear Fluorescent Transition (RESOLFT), Total Internal Reflection Fluorescence Microscope (TIRFM), Fluorescence-PALM (FPALM), Stochastical Optical Reconstruction Microscopy (STORM), Fluorescence Imaging with One-Nanometer Accuracy (FIONA), and combinations thereof. For examples: Chi, 2009 “Super-resolution microscopy: breaking the limits,” Nature Methods 6(1): 15-18; Blow 2008, “New ways to see a smaller world,” Nature 456:825-828; Hell, et al, 2007, “Far-Field Optical Nanoscopy,” Science 316: 1153; R. Heintzmann and G. Ficz, 2006, “Breaking the resolution limit in light microscopy,” Briefings in Functional Genomics and Proteomics 5(4):289-301; Garini et al., 2005, “From micro to nano: recent advances in high-resolution microscopy,” Current Opinion in Biotechnology 16:3-12; and Bewersdorf et al, 2006, “Comparison of I⁵M and 4Pi-microscopy,” 222(2): 105-1 17; and Wells, 2004, “Man the Nanoscopes,” JCB 164(3):337-340.

In some embodiments, electron microscopes (EM) are used for imaging.

In some embodiments, an imaging step detects a gene segment. In some embodiments, an imaging step localizes a gene segment. In some embodiments, an imaging step provides three-dimensional spatial information of a gene segment. In some embodiments, an imaging step quantifies a gene segment. By using multiple contacting and imaging steps, provided methods are capable of providing spatial and/or quantitative information for a large number of gene segments in surprisingly high throughput. For example, when using F detectably different types of labels, spatial and/or quantitative information of up to F_{N} gene segments can be obtained after N contacting and imaging steps.

Certain techniques for imaging are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

In some embodiments, the method comprises analyzing cell size and shape, markers, immunofluorescence measurements, or any combinations thereof.

In some embodiments, the method comprises using super-resolution microscopy to image the barcode.

### EXAMPLES

The following non-limiting methods are provided to further illustrate the embodiments of the invention disclosed herein. It should be appreciated by those of skill in the art that the techniques disclosed in the examples that follow represent approaches that have been found to function well in the practice of several embodiments of the invention, and thus be considered to constitute examples of modes for its practice. However, those of skill in the art should, in light of the present disclosure, appreciate that many changes can be made in the specific embodiments that are disclosed and still obtain a like or similar result without departing from the spirit and the scope of the invention.

### Methods

### Coverslip Functionalization and Flow Cell Preparation

Glass coverslips (Epredia #152460) were immersed in RNase-free water (Invitrogen #10977-023) then immersed in 100% ethanol (Koptec #V1016). Coverslips were dried for 5 minutes in a 90° C. oven (Bellco Glass Inc. #7930-00110) then plasma cleaned on “high” for 5 minutes (Harrick Plasma #PDC-001). Cleaned coverslips were coated overnight in a humidified chamber at room temperature with 0.1 mg/mL 70,000-150,000 MW poly-D-lysine (Sigma-Aldrich #P6407-5MG) in water. Coated coverslips were rinsed again with RNase-free water, dried under nitrogen gas, stored at 4° C., and used within 1 week.

Alternatively, to improve adherence of tissue sections, plasma-cleaned coverslips were coated for 30 minutes at room temperature with a solution of 1% (w/v) 3-aminopropyltriethoxysilane (APTS) (Thermo Scientific #80370), 9.9 mM acetic acid (Sigma-Aldrich #A6283-500ML), and 89.1% ethanol. APTS-coated coverslips were rinsed 3 times in 100% ethanol and dried for 10-30 minutes in a 90° C. oven. Dried APTS-coated coverslips were then coated overnight in a humidified chamber at room temperature with 0.1 mg/mL >300,000 MW poly-D-lysine (Sigma-Aldrich #P1024) in 100 mM borate buffer (Thermo Scientific #28341). Coated coverslips were rinsed again with RNase-free water, dried under nitrogen gas, stored at 4° C., and used within 1 week.

Flow cells were prepared using glass microscope slides with drilled inlet and outlet holes. Slides were plasma cleaned on “high” for 7 minutes, and laser cut adhesive was aligned and adhered to the slide creating a channel between the holes. Flow cells were stored at room temperature before affixing to coverslips.

### Human T Cell Culture and Fixation

Cryovials of Jurkat E6-1, Jurkat JM22, and Jurkat CH7C17 T leukemic cell lines were thawed, centrifuged at 300×g for 5 minutes, and cultured in RPMI 1640 with GlutaMAX and 25 mM HEPES (Gibco #72400-047)+10% heat-inactivated FBS (Corning #35-011-CV). Media for Jurkat JM22 was additionally supplemented with 0.4 mg/mL Geneticin (G418 Sulfate) (Gibco #10131-035) for selection of transduced cells containing the JM22 TRB TCR construct. Lymphocyte cultures were maintained at 37° C. and 5% CO2 in T25 culture flasks (VWR #10062-872) at a density of 105-106 cells/mL.

Lymphocyte cultures were counted on a hemocytometer (Bulldog Bio #DHC-NO1), and 100,000 cells/coverslip were pelleted by centrifugation at 300×g for 5 minutes. The cell pellet was resuspended at 2,000 cells/μL, and 100,000 cells in 50 μL was added to the center of a 70,000-150,000 MW poly-D-lysine-coated glass coverslip. Cells were immediately fixed by adding 200 μL 4% paraformaldehyde (PFA) (Thermo Scientific #28906) in 1×PBS (Invitrogen #AM9625) and incubating for 5 minutes at room temperature. Fixed coverslips were rinsed with 1×PBS and 75% ethanol. Coverslips with fixed lymphocytes were dried under nitrogen and a flow cell was installed for immediate use. Alternatively, coverslips with fixed lymphocytes were stored at −20° C. in 70% ethanol for up to 3 months.

### Oligonucleotide Probe Design

Oligonucleotide probes were designed in JupyerLab v3.4.4 running Python v3.9.13. Human T cell receptor (TCR) gene sequences were retrieved from the international ImMunoGeneTics information system (IMGT) gene database (IMGT/GENE-DB).47 TCR gene sequences were read in FASTA format and filtered for variable (V), joining (J), and constant (C) genes of TRA, TRB, TRG, and TRD. The gene list was further filtered to remove non-functional pseudogenes and to only keep the most common allele for each gene. To account for random nucleotide deletion at TCR junctional sites, 4 nucleotides were removed from the 3′ end of V sequences and the 5′ end of J sequences.

Antisense oligonucleotide probes were designed as described in Eng et al. without filtering by % GC content. In addition, the constant regions of each TCR gene were included for colocalization as well as the following genes for T cell subtyping in the skin: CD3E, CD4, CD8A, SELL (L-selectin), CCR7, ITGAE (CD103), CD69, CXCR3, GZMB, GZMA, PRF1, and ITGA1 (CD49A). Up to 10 probes per gene were generated with a length of 35 nt and spacing of 2 nt. Each probe was manually checked, and only gene-specific probes were kept. Due to sequence homology between TCR genes, gene-specific probes could not be generated for a handful of TCR genes. Instead, the probe list contains probes complementary to the following pairs of TCR genes: TRAV38-1+TRAV38-2/DV8, TRAV8-2+TRAV8-4, TRBC1+TRBC2, TRBV10-1+TRBV10-2, TRBV12-3+TRBV12-4, TRBV5-5+TRBV5-7, TRBV6-5+TRBV6-6, TRBV7-6+TRBV7-7, TRGJ1+TRGJ2, and TRGV3+TRGV5. Unique probes were unable to be generated for the following 5 genes: TRBV24/OR9-2, TRBV29/OR9-2, TRBV6-3, TRBV6-9, and TRBV7-8.

Genes were barcoded across 16 total rounds of hybridization with 3 fluorescent channels per round (647 nm, 561 nm, and 488 nm). Within-channel barcoding was used for the first 12 hybridization rounds. TRAV and TRGV were assigned to the 647 nm channel, TRBV and TRDV were assigned to the 561 nm channel, and TRAJ and TRGJ were assigned to the 488 nm channel. Across-channel barcoding was used for the last 4 hybridization rounds to assign barcodes for TRBJ, TRDJ, TCR constant genes, and T cell subtyping genes. Within-channel barcoding used 4 pseudocolors with 3 barcoding rounds for a total of 64 possible codes per channel. Across-channel barcoding used 6 pseudocolors with 2 barcoding rounds for a total of 36 possible codes. Codes were randomly assigned for each gene.

The final oligonucleotide probe sequences were stitched together using selected antisense probe sequence(s) and amplifiers according to the randomly assigned pseudocolor barcode. Each probe contains the following primers for probe generation by PCR and in vitro transcription: forward primer, 5′-GCCCCATCATGTGCCTTTC-3′; reverse primer, 5′-CTATAGTGAGTCGTATTACCGGCC-3′. The reverse primer represents just the T7 RNA polymerase binding site for in vitro transcription, which allows for probe circularization by hybridization and ligation of a splint oligonucleotide (5′-CATGATGGGGCGCGTAGAGTG-3′) complementary to the 5′ end of the forward primer and the 3′ universal sequence (5′-CACTCTACG-3′). The final sequence of within-channel probes is as follows: 5′-[Forward Primer]-[Amplifier A]-CAA-[Gene-Specific Probe]-AAC-[Amplifier B]-CTATAC-[Amplifier C]-AAC-[Universal]-[Reverse Primer]-3′. The final sequence of across-channel probes is as follows: 5′-[Forward Primer]-[Amplifier A]-CAA-[Gene-Specific Probe]-AAC-[Amplifier B]-AAC-[Universal]-[Reverse Primer]-3′.

### Probe Generation

Custom oligonucleotide probe pools were synthesized by Twist Bioscience (South San Francisco, CA) with a yield of >0.2 fmol average per oligonucleotide. The lyophilized probes were resuspended in water at 1 ng/μL and shaken at 9 Hz in a 37° C. ThermoMixer (Eppendorf #5436) for 5 minutes to fully dissolve. Probes were first amplified by limited cycle PCR. The PCR reaction mixture was prepared by mixing 2 μL resuspended oligonucleotide pool, 2.5 μL each of 10 μM forward (5′-GCCCCATCATGTGCCTTTCC-3′) and reverse (5′-GGCCGGTAATACGACTCACTATAGG-3′) primers (Integrated DNA Technologies), 25 μL KAPA HiFi HotStart ReadyMix (Roche #7958927001), and 18 μL water. The reaction mixture was split into 2 tubes and run on a thermocycler (Bio-Rad #1851148) with the following settings: initial denaturation at 98° C. for 4 minutes; 13 cycles of denaturation at 98° C. for 20 seconds, annealing at 69° C. for 20 seconds, and extension at 72° C. for 20 seconds; and a final extension at 72° C. for 2 minutes. The PCR product was purified using the QIAquick PCR purification kit (QIAGEN #28104) with a final elution in 40 μL water. The concentration of dsDNA was determined by spectrophotometry using a NanoDrop One (Thermo Scientific).

The probe pool was further amplified using in vitro transcription to produce many copies of RNA from each strand of DNA. The number and volume of reactions were determined using 0.5-1 μg DNA/reaction and 30 μL per reaction. The final in vitro transcription reaction mixtures were prepared to contain 0.5-1 g DNA/reaction, 5,000 units/mL T7 RNA polymerase (New England Biolabs #M0251S), 1× RNAPol reaction buffer (New England Biolabs #B9012S), 10 mM each rNTP (Thermo Scientific #R0481), 1 U/μL RNasin Plus RNase inhibitor (Promega #N261A), and 2.5 U/mL inorganic pyrophosphatase (New England Biolabs #M0361). Tubes were incubated in a 37° C. thermocycler for 16 hours. To remove the dsDNA template, tubes were incubated at 37° C. for 30 minutes with 80 U/mL DNase (New England Biolabs #M0303). RNA was then purified by adding 180 μL paramagnetic solid phase reversible immobilization beads (Bulldog Bio #CNGS050) and incubating at room temperature for 5-10 minutes. Tubes were then placed in a magnetic rack (Invitrogen #12321D) for 5 minutes to pellet the beads. Supernatant was removed and the pellet was washed 3 times for 30s with 500 L freshly made 75% ethanol, being careful not to resuspend or disturb the pellet. The pellet was then air dried for 5-10 minutes to remove leftover ethanol. Tubes were removed from the magnetic rack and purified RNA was eluted from the beads with 100 μL water and shaken at 9 Hz in a 37° C. ThermoMixer for 5 minutes. Tubes were returned to the magnetic rack, and beads were allowed to pellet for 5 minutes. The supernatant was transferred to a new tube, and the concentration of RNA was determined by NanoDrop.

Next, the RNA was immediately reverse transcribed to generate the final ssDNA probe. First, the concentration of RNA and the expected strand length were used to determine the total amount of RNA in nmols. The total reaction volume was determined as 40 μL/nmol RNA.

Reverse transcription reactions were prepared to contain 1.5 M RNA, 37.5 M forward primer, 0.4 U/μL RNasin Plus RNase inhibitor (Promega #N261A), 1 U/mL inorganic pyrophosphatase (New England Biolabs #M0361, 1× RT Buffer (Thermo Scientific), 3 mM each dNTP (Thermo Scientific #R0182), and 1 μL/nmol RNA Maxima H Minus Reverse Transcriptase (Thermo Scientific #EP0752). Tubes were incubated in a thermocycler with the following settings: 55° C. for 2 hours, 50° C. for 2 hours, and 85° C. for 5 minutes. Next, alkaline hydrolysis was used to remove RNA by adding 1M sodium hydroxide (Macron Fine Chemicals #7708-10) for a final concentration of 250 mM and incubated at 65° C. for 20 minutes. The solution was neutralized by adding 1M acetic acid (Sigma-Aldrich #A6283-500ML) for a final concentration of 250 mM. Gel electrophoresis was performed to evaluate probe size and purity. The gel consisted of 2% agarose (VWR #0710) in 1× TBE (Invitrogen #AM9863) with 1:10,000 SYBR Safe (Thermo Scientific #S33102). ssDNA probes were mixed with DNA loading dye (Thermo Scientific #R1161), loaded alongside a 50 bp DNA ladder (New England Biolabs #N0556S), and run in 1× TBE in a horizontal electrophoresis chamber (Thermo Scientific #B1A-BP) at 150V for 30 minutes (Bio-Rad #1645050) before observation and imaging under blue light transillumination (Invitrogen #G6600). Next, 0.1 volumes of 3M sodium acetate (Invitrogen #AM9740) and 4 volumes of ethanol were added to each tube and allowed to precipitate at −20° C. for 15 minutes minimum. Probes were pelleted by centrifugation at 16,000×g for 30 minutes at 4° C. Supernatant was removed, freshly made 75% ethanol was added without disturbing the pellet, and tubes were centrifuged at 16,000×g for 5 minutes at 4° C. Supernatant was removed, and the pellet was allowed to air dry. Probes were resuspended in 100 μL water and a final paramagnetic solid phase reversible immobilization bead purification was performed using 1.8 volumes of beads. Final, full-length ssDNA oligonucleotide probes were eluted in 50 μL water and the concentration was determined by NanoDrop. If the probe concentration was determined to be less than 4 nM/oligo, probes were lyophilized and resuspended at the correct volume using a SpeedVac (Thermo Scientific).

### Probe Hybridization and Amplification

After installation of the flow cell, samples were rehydrated by flowing in 1×PBS for 3 minutes three times at room temperature. Next, samples were post-fixed in 7.5 mM PEGylated bis(sulfosuccinimidyl)suberate (BS(PEG)5) (Thermo Scientific #A35396) in 10% dimethyl sulfoxide (DMSO) (Invitrogen #D12345) and 1× PBS for 30 minutes at room temperature. Samples were washed with 1×PBS, then charge quenching was performed using two 1-hour-long incubations at room temperature with 50 M methacrylic acid N-hydroxysuccinimide ester (Sigma-Aldrich #730300) in 5% DMSO and 1× PBS. Samples were washed 3 times with 40% (v/v) formamide (Millipore #4650), 0.1% triton X-100, 2× saline-sodium citrate (SSC) (Invitrogen #15557-044) wash buffer prior to probe hybridization.

Primary probe hybridization was performed at 37° C. for 24-48 hours at 2 nM/oligo in 50% formamide, 0.1% (w/v) 500,000 MW dextran sulfate (Millipore #3710-50GM), and 2×SSC hybridization buffer. Afterwards, the sample was washed twice with 55% formamide, 0.1% triton X-100, 2×SSC wash buffer for 30 minutes at 37° C. Samples were then rinsed 3 times with 2×SSC and 3 times with 0.1% triton X-100 in 1×PBS (PBST) prior to probe ligation.

Smfish Probes for TRBJ2-7 were Amplified by Hybridization Chain Reaction

Hybridized probes were circularized by ligation using a splint oligonucleotide complementary to the 5′ end of forward primer and the 3′ universal sequence. First, the sample was washed with 1× Quick Ligase Buffer (New England Biolabs #B2200S). Next, the sample was incubated at room temperature for 1 hour with 100 M splint oligonucleotide (Integrated DNA Technologies) and 1:10 Quick Ligase (New England Biolabs #M2200S) in 1× Quick Ligase Buffer. Afterwards, samples were washed 3 times with PBST. To remove non-circularized probes, samples were rinsed with 1× rCutSmart buffer (New England Biolabs #B6004S) then incubated with 400 units/mL exonuclease I (New England Biolabs #M0293S), 600 units/mL RecJ_{f} exonuclease (New England Biolabs #M0264S), 400 units/mL SUPERase RNase inhibitor (Invitrogen #AM2694), and 5% DMSO in 1× rCutSmart Buffer for 30 minutes at 37° C. Samples were washed 3 times with 2×SSC and stored at 4° C. in 2×SSC prior to circularized probe amplification.

Circularized probes were amplified in a custom automated microfluidics system using sequential rounds of hybridization and ligation of alternating amplifier oligonucleotides. Amplifier hybridization was performed at 37° C. for 45 minutes at 100 nM/oligo in 10% formamide, 10% (w/v) 40,000 MW dextran sulfate (Sigma-Aldrich #42867-25G), 1% (w/v) 500,000 MW dextran sulfate, 0.1% triton X-100, and 2×SSC. Next, the sample was washed with 10% formamide, 0.1% triton X-100, 2×SSC wash buffer. Afterwards, ligation was performed at room temperature with a solution of 120,000 units/mL T3 DNA ligase (New England Biolabs #M0317L), 200 units/mL exonuclease I, 300 units/mL RecJ_{f} exonuclease, 200 units/mL SUPERase RNase inhibitor, 1 mM ATP (New England Biolabs #P0756S), 300 mM sodium chloride, 7.5% PEG 6000 (Rigaku Reagents #1008061) and 1× rCutSmartBuffer. Six total rounds of amplification were performed.

### Automated Sequential Fluorescence In Situ Hybridization and Confocal Microscopy

Sequential fluorescence in situ hybridization (seqFISH) was performed with custom automated microfluidics and a microscope (Leica #DMi8) with a spinning disc confocal unit (Andor #CSU-W). Images were taken with a 40× oil immersion lens (Leica #506329, Leica #11513859) using a Zyla sCMOS camera (Andor). To correct for chromatic aberration, the sample was first incubated with 1:100 TetraSpeck beads (Invitrogen #T7280) in 2×SSC for 10 minutes at room temperature, washed 3 times with 1×PBS, and fixed with 4% PFA in 1×PBS for 15 minutes at room temperature. In each round of seqFISH, fluorescently labeled readout probes were hybridized at 100 nM/probe in 10% formamide, 100 mg/mL 6,500-10,000 MW dextran sulfate (Sigma-Aldrich #D4911), and 2×SSC for 15 minutes at room temperature. The sample was then washed with 10% formamide, 0.1% triton X-100, 2×SSC wash buffer, rinsed with 2×SSC, and incubated with 3 g/mL DAPI (Sigma-Aldrich #D8417-5MG) for 1 minute at room temperature. The sample was rinsed again with 2×SSC before a solution of 50 mM Tris HCl (Invitrogen #15568-025), 1 mM Trolox (Sigma-Aldrich #238813), 10% glucose (Sigma-Aldrich #G7528-250G), 1× catalase (Sigma-Aldrich #C3155-50MG), 1 mg/mL glucose oxidase (Sigma-Aldrich #G2133-10KU), and 2×SSC was applied for imaging. Multiple fields of view with Z-stacks were imaged in 647 nm, 561 nm, 488 nm, and 405 nm. After imaging, readouts were removed with 55% formamide, 0.1% triton X-100, 2×SSC wash buffer for 5 minutes at room temperature. The sample was then rinsed with 2×SSC before hybridizing and imaging the subsequent sets of readouts.

### Image Processing

Imaging processing was performed using the Caltech Resnick High Performance Computing Center. Microscopy images were processed and decoded using PyFISH, a seqFISH image processing implementation written in Python by Katsuya Colon and Arun Chakravorty. First, images in separate hybridization rounds were aligned using the DAPI (405 nm) channel, and chromatic aberration was corrected using the TetraSpeck bead locations. Images were background-subtracted and fluorescent dot locations were detected in each encoding channel using the DAOFIND algorithm implemented in Photutils. Dot location and hybridization round were decoded to determine the encoded gene at each diffraction limited point. Counts per cell were determined using cell masks segmented using the Cellpose algorithm.

### Analysis

Signal-to-noise ratio of smFISH and HCR images was calculated in ImageJ vi.51s by averaging signal from fluorescent dots and dividing by the average background signal. Hierarchically clustered heatmaps of single cell spleen data was graphed in JupyerLab v3.4.4 running Python v3.9.13 using the seaborn clustermap function. Single cell TCR sequencing data of vitiligo lesional suction blisters was collected and pre-processed by Erica Katz and John Harris at the University of Massachusetts Chan Medical School. TCR data was extracted in Python using pyTCR and graphed in R v4.2.0 using circlize. TCR V and J gene segment usage empirical cumulative density function (ECDF) was graphed in Python using iqplot.

### Experiment 1

### TCR Seqfish Allows Imaging of Clonotype-Specific TCR Transcripts in Single Cells

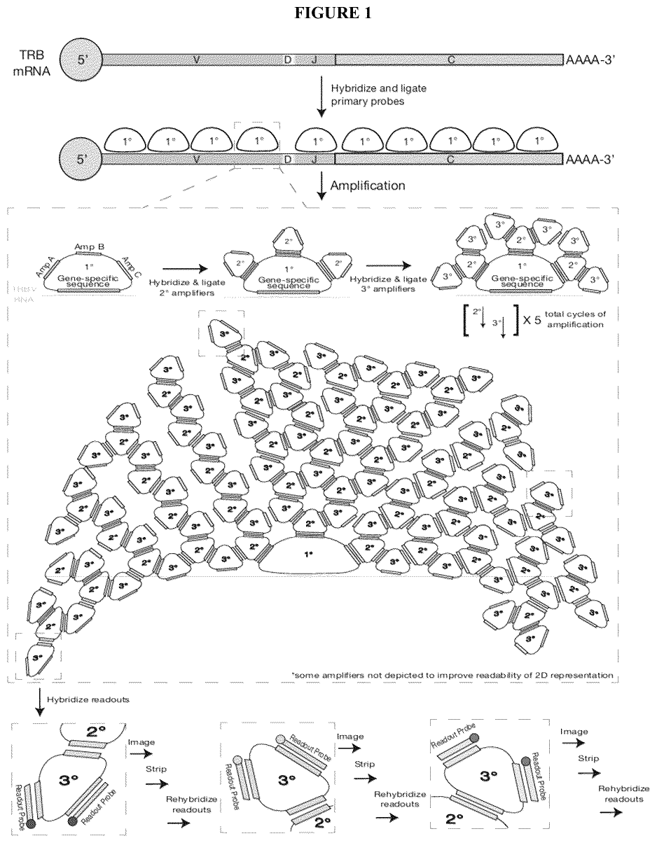

Although fluorescently-labeled oligonucleotide probes have been used for over 20 years for imaging individual mRNA molecules. single molecule FISH (smFISH) has not previously been demonstrated for individual TCR mRNA transcripts. Oligonucleotide probes were designed targeting the specific gene rearrangement of the influenza A virus matrix protein-specific JM22 TCR_{β} chain (TRBV19, TRBJ2-7, TRBC2). Importantly, probes for each gene segment (V, J, and C) were designed such that each segment is read out in a different fluorescent channel (561 nm, 647 nm, and 488 nm, respectively), thereby using colocalization of fluorescent points in all 3 channels for identification of whole JM22 TRB mRNA transcripts (FIG. 1A). Indeed, intact mRNA transcripts were positively identified using smFISH in a Jurkat cell line expressing JM22 TRB (FIG. 1i). These JM22 TRB-specific probes did not colocalize in the parental Jurkat E6.1 cell line (FIG. 1C), demonstrating that smFISH targeting TCR gene arrangements is clonotype specific.

Differences in TCR gene segment length and TCR gene family sequence homology limits the maximum number of probes that can be designed for any given TCR gene. In the case of JM22 TRB, 35 nt probe binding sites were used, resulting 10 probes for TRBV19, 1 probe for TRBJ2-7, and 13 probes for TRBC2. Differing numbers of probes affects the number of fluorescent dye molecules that can be localized at the TCR mRNA, which is particularly evident in the high signal-to-noise ratio of the singularly-labelled J segment in JM22 TRB transcripts (FIG. 1i). Signal amplification by hybridization chain reaction (HCR) was performed demonstrating that amplification of probe signal from short J gene segments improves signal-to-noise ratio and is useful in detection of specific TCR mRNAs (FIG. 1D).

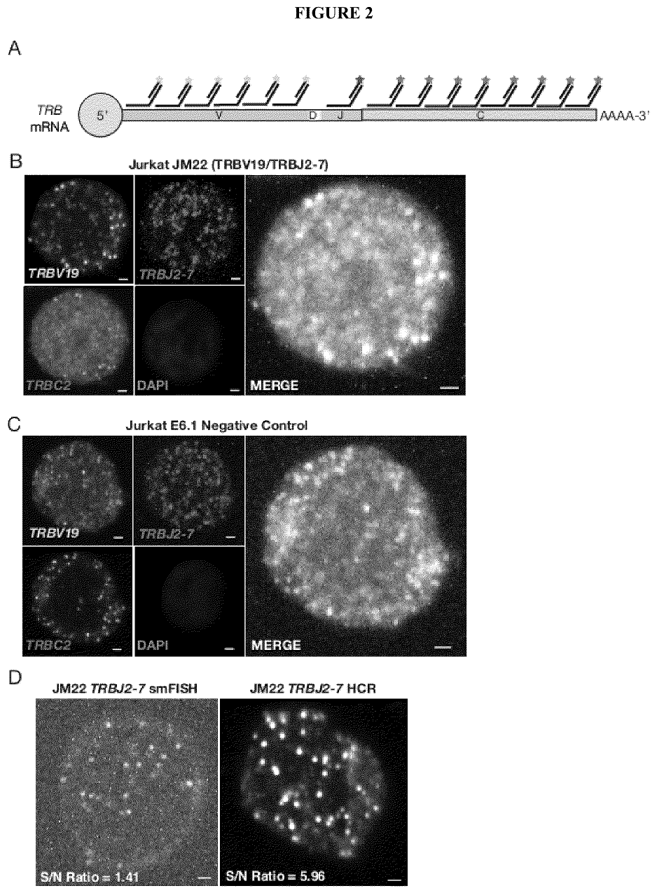

Next, a generalized set of oligonucleotide probes were designed to allow for identification of human V, J, and C gene segments for TRA, TRB, TRG, and TRD. These probes were multiplexed such that the identity of each diffraction-limited point can be decoded using sequential rounds of readout hybridization to generate a temporal pseudocolor barcode (FIG. 2A). Using a programmable sequential amplification strategy based on clampFISH, multiplexing the detection of TCR probes was performed by building multiple amplifier trees on the same primary probe with each amplifier corresponding to a barcoding round for a specific gene (Figure Si). Similar to results using smFISH, JM22 TRB gene segments were decoded using this probe pool in JM22 Jurkat cells (FIG. 2B).

### Experiment 2

### TCR Repertoire Analysis by Seqfish

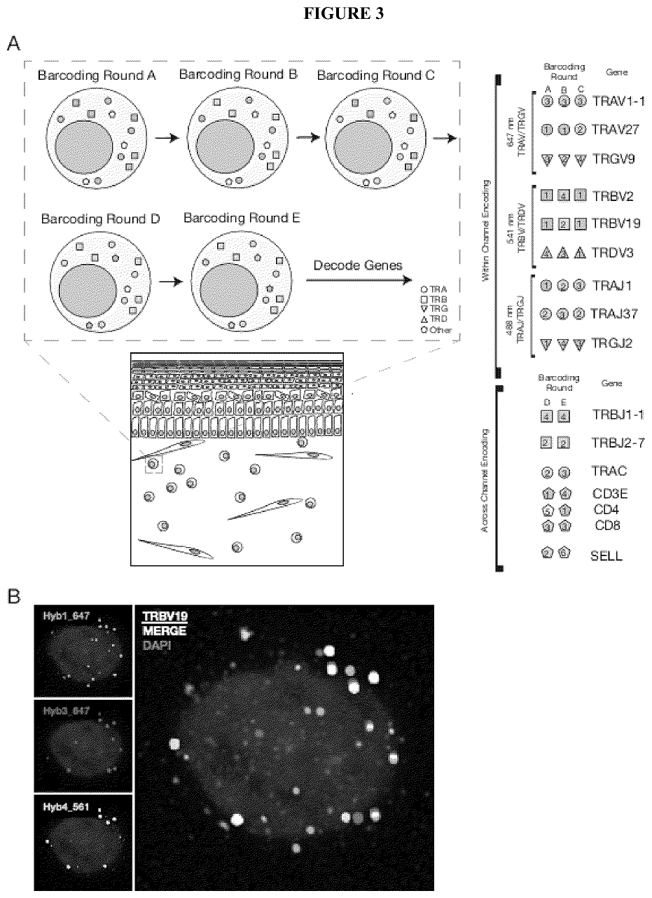

Next, TCR seqFISH was tested in fresh frozen human spleen to demonstrate its ability to characterize the T cell repertoire in polyclonal tissue sections. The red pulp surrounding a germinal center was selected for imaging because this area is rich in migratory T cells recirculating in the spleen (FIG. 3A). Many different V gene transcripts from the spleen were decoded, in line with T cell polyclonality in the spleen (FIG. 3B). Importantly, the decoding rate for unused barcodes was lower than those encoding the majority of V genes, indicating that this approach has low false positives.

### Experiment 3

### Spatial Mapping of CDR3 Sequences Using TCR Seqfish Combined with NGS

Using our oligonucleotide probe pool targeting human TCR gene segments, it is possible to evaluate lymphocyte clonality in fixed tissues by imaging, defining each clone by its combination of V and J TCR gene segments (αβ or γδ). Although V/J pairing information is sufficient for the identification of T cell clones, it lacks the CDR sequence-level data which is most useful in predicting epitope specificity. To address this limitation, a method was developed where post hoc bulk sequencing can be performed after TCR seqFISH to map CDR sequences back to individual lymphocytes in super resolution images.

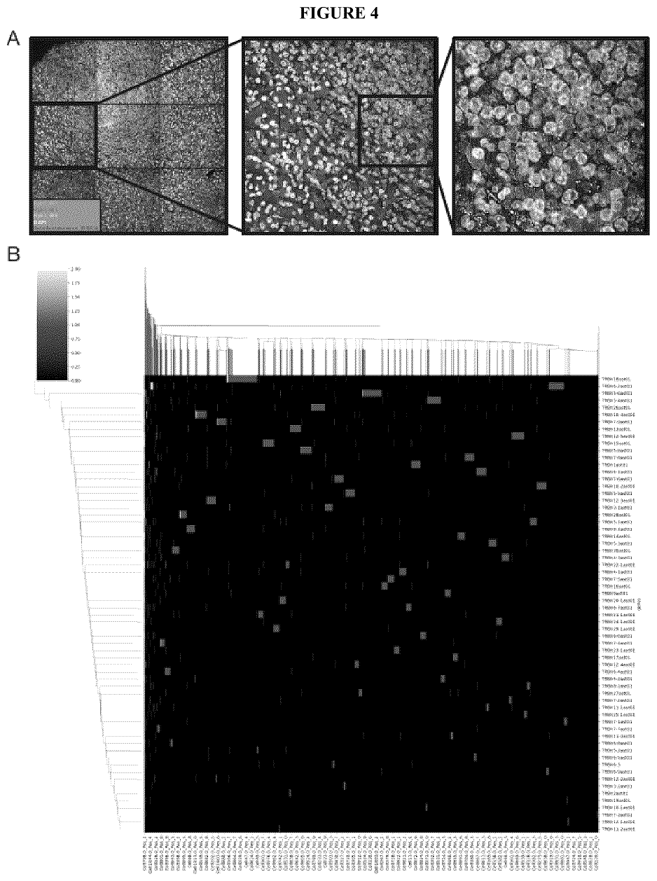

Combining bulk sequencing of recombined TCR genes with TCR seqFISH relies upon using unique V/J gene segment pairing to match CDR sequences to individual clones in situ. To assess the feasibility of this approach, single cell TCR sequencing data of T cells in the skin of a patient with vitiligo, an autoimmune disease characterized by loss of melanocytes and skin depigmentation, was analyzed. Analysis of V/J gene pairing for each CDR3 sequence demonstrated that the majority of T cell clones sequenced had unique V/J pairing (FIG. 4), supporting the feasibility of using bulk sequencing to map CDR3 sequences to TCR seqFISH images. Notably, clonotypes with unique V/J pairing were observed at a higher rate in TRA than in TRB, likely due to the lower number of TRBJ genes, which makes it more likely that 2 different clones share the same V/J gene pairing. The rate of shared V/J genes among unrelated clones is anticipated to be less of an issue with post hoc sequencing from a single fixed section due to lower absolute numbers of clones in thin sections.

### Experiment 4

### Chemical “Read-and-Anchor Split” Amplification

Assays employing a limited set of primary probes targeting human TCR region (TRBV19, TRBJ7-2, and TRBC2) mRNA were performed using Copper(I)-catalyzed Azide-Alkyne Cycloaddition (CuAAC; Process A) and 4-benzoylbenzoic acid (BBA)-mediated photocrosslinking (Process B). The jurkat JM22 cell sample preparation followed the workflow outlined in Hu, X. et al. 2015.

### Probe Design:

Table 1 lists the probes used in this experiment.

Primary probes: 10, 1, 13 probes were designed separately to target human TCR region TRBV19, TRBJ7-2, and TRBC2. The RNA binding region length was 35 nucleotides. Each probe contains one RNA binding site and two secondary amplifier-anchor binding sites. Specially, the amplifier-anchor binding sites on the TRBJ7-2 primary probe were repeated for 3 times.

Anchors: Anchor oligonucleotides consisted of 15 nucleotides DNA modified with a 5′ alkyne and a 3′ BBA group. Oligos with 5′-alkyne and 3′-amine were purchased from IDT. The 3′-BBA modification was introduced using 4-benzoylbenzoic acid N-succinimidyl ester, followed by purification via RP-HPLC. Distinct anchor sequences were used for secondary and tertiary amplification stages.

Amplifiers: Each amplifier contained (i) a sequence complementary to the amplification site of the preceding probe, (ii) two repeats of its orthogonal amplification site, and (iii) the two corresponding anchor sites. N⁶-(6-azido)hexyl-dATP was incorporated at the 3′ end through TdT-mediated extension.

Hybridization and Split-Assay CuAAC Reaction: After charge quenching, flow in the hybridization buffer containing 5 nM/oligo primary probe, 0.1 mg/ml yeast tRNA, and 2 M polyTTG (200 nucleotides) in 10% 40 kDa Dextran Sulfate and 25% formamide. The sample was incubated at 37° C. for 18 hours.

For split-primary-probe assay, a primary CuAAC step was required. Fresh click buffer (2×SSC, 75 μM CuSO₄, 375 μM BTTAA, 0.25% Triton X-100, 5% DMSO, and 3 mM (+)-sodium L-ascorbate) was prepared and applied for 1 h at 37° C. 60% formamide wash was performed afterwards at 37° C. to remove nonspecific sticking.

Amplification Procedure: Amplification was conducted over 12 sequential rounds. Generally, each round consisted of:
Hybridization of secondary or tertiary amplifiers (50 nM) and anchors (75 nM) for 1 h at 37° C.;
Apply 10% formamide wash buffer to remove nonspecific binding.
Incubation with CuAAC click buffer for 60 min at 37 C;
Apply 30% formamide wash buffer to remove nonspecific binding.
UV irradiation at 365 nm (~130 mW) for 10 min at RT to induce photocrosslinking.
Removal of unligated/photocrosslinked oligonucleotides by applying 60% formamide wash for 10 min.

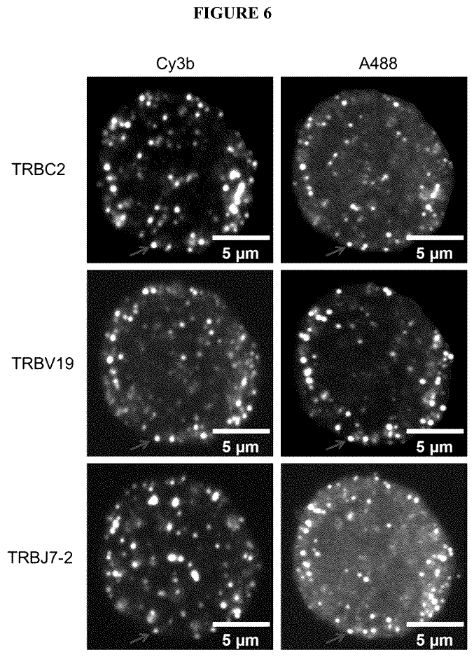

Amplification and imaging: After amplification, probes targeting each region were recognized by two fluorescent readout probes incorporating fluorophores Cy3b and A488. 3 TCR regions were readout out in 3 sequential rounds. Only dots colocalized across all six channels were considered real signals. During image acquisition, the Cy3b, and A488 channels were exposed for 2000 ms respectively. The fluorescent signals observed across the three channels demonstrated strong signal intensity and colocalization between channels, as shown in FIG. 6

TABLE 1

| Probe | SEQUENCE |
|---|---|
| TRBC2 probes: | GCCCCATCATGTGCCTTTCcTTGACGAAGCCTGACACTCAAGGCCGGTATaCTCAAACACAGCGAC |
| | CTTGGGTGGGAACACGTTTTaTTGACGAAGCCTGACTGATACGCGAACTGTaCCCTATAGTGAGTC |
| | GTATTACCGGCC |
| | GCCCCATCATGTGCCTTTCcTTGACGAAGCCTGACACTCAAGGCCGGTATaTGGCCTTTTGGGTGT |
| | GGGAGATCTCTGCTTCTGATaTTGACGAAGCCTGACTGATACGCGAACTGTaCCCTATAGTGAGTCG |
| | TATTACCGGCC |
| | GCCCCATCATGTGCCTTTCcTTGACGAAGCCTGACACTCAAGGCCGGTATaCACCTCCTTCCCATTC |
| | ACCCACCAGCTCAGCTCCAaTTGACGAAGCCTGACTGATACGCGAACTGTaCCCTATAGTGAGTCG |
| | TATTACCGGCC |
| | GCCCCATCATGTGCCTTTCcTTGACGAAGCCTGACACTCAAGGCCGGTATaCTCAGGCAGTATCTGG |
| | AGTCATTGAGGGCGGGCTGaTTGACGAAGCCTGACTGATACGCGAACTGTaCCCTATAGTGAGTCG |
| | TATTACCGGCC |
| | GCCCCATCATGTGCCTTTCcTTGACGAAGCCTGACACTCAAGGCCGGTATaCGTAGAACTGGACTT |
| | GACAGCGGAAGTGGTTGCGGaTTGACGAAGCCTGACTGATACGCGAACTGTaCCCTATAGTGAGT |
| | CGTATTACCGGCC |
| | GCCCCATCATGTGCCTTTCcTTGACGAAGCCTGACACTCAAGGCCGGTATaGCCCTATCCTGGGTCC |
| | ACTCGTCATTCTCCGAGAGaTTGACGAAGCCTGACTGATACGCGAACTGTaCCCTATAGTGAGTCGT |
| | ATTACCGGCC |
| | GCCCCATCATGTGCCTTTCcTTGACGAAGCCTGACACTCAAGGCCGGTATaAGGATGGTGGCAGAC |
| | AGGACCCCTTGCTGGTAAGAaTTGACGAAGCCTGACTGATACGCGAACTGTaCCCTATAGTGAGTC |
| | GTATTACCGGCC |
| | GCCCCATCATGTGCCTTTCcTTGACGAAGCCTGACACTCAAGGCCGGTATaGGCATACAAGGTGGC |
| | CTTCCCTAGCAAGATCTCATaTTGACGAAGCCTGACTGATACGCGAACTGTaCCCTATAGTGAGTCG |
| | TATTACCGGCC |
| | GCCCCATCATGTGCCTTTCcTTGACGAAGCCTGACACTCAAGGCCGGTATaGGTTTTGGAGCTAGC |
| | CTCTGGAATCCTTTCTCTTGaTTGACGAAGCCTGACTGATACGCGAACTGTaCCCTATAGTGAGTCG |
| | TATTACCGGCC |
| | GCCCCATCATGTGCCTTTCcTTGACGAAGCCTGACACTCAAGGCCGGTATaGGAGAATCCTGGGTG |
| | AGGATGAAGAATGACCTGGGaTTGACGAAGCCTGACTGATACGCGAACTGTaCCCTATAGTGAGTC |
| | GTATTACCGGCC |
| | GCCCCATCATGTGCCTTTCcTTGACGAAGCCTGACACTCAAGGCCGGTATaGAATCACTTTTAGGAA |
| | CACAGATTGGGAGCAGGTAaTTGACGAAGCCTGACTGATACGCGAACTGTaCCCTATAGTGAGTCG |
| | TATTACCGGCC |
| | GCCCCATCATGTGCCTTTCcTTGACGAAGCCTGACACTCAAGGCCGGTATaAAGTATTCATGTAAGT |
| | AGGAGATGAGAAGCAGAGTaTTGACGAAGCCTGACTGATACGCGAACTGTaCCCTATAGTGAGTC |
| | GTATTACCGGCC |
| | GCCCCATCATGTGCCTTTCcTTGACGAAGCCTGACACTCAAGGCCGGTATaGGGGTTGGGAGCTCA |
| | ATCTTCAGGGAAACAGAAAAaTTGACGAAGCCTGACTGATACGCGAACTGTaCCCTATAGTGAGTC |
| | GTATTACCGGCC |
| TRBJ2-7 probes: | GCCCCATCATGTGCCTTTCcTTGACGAAGCCTGACCATGAATTCTCGCCAaTTCTCCTGGCCTAATGT |
| | GCAGAGTTTGTCAGAGGCaTTGACGAAGCCTGACCACTCGTACAGCGTTaCCCTATAGTGAGTCGT |
| | ATTACCGGCC |
| | GCCCCATCATGTGCCTTTCcTTGACGAAGCCTGACCATGAATTCTCGCCAaTTGGAAGAGAATGAG |
| | AAAAGAGACTTGGCTCGGGGaTTGACGAAGCCTGACCACTCGTACAGCGTTaCCCTATAGTGAGT |
| | CGTATTACCGGCC |
| | GCCCCATCATGTGCCTTTCcTTGACGAAGCCTGACCATGAATTCTCGCCAaCAGCAGAGCACCTGG |
| | TTGCTCATAGTGCAAAGGGGaTTGACGAAGCCTGACCACTCGTACAGCGTTaCCCTATAGTGAGTC |
| | GTATTACCGGCC |
| | GCCCCATCATGTGCCTTTCcTTGACGAAGCCTGACCATGAATTCTCGCCAaTACTTTGGGGACTGAG |
| | TGATTCCACCATCCACGGTaTTGACGAAGCCTGACCACTCGTACAGCGTTaCCCTATAGTGAGTCGT |
| | ATTACCGGCC |
| | GCCCCATCATGTGCCTTTCcTTGACGAAGCCTGACCATGAATTCTCGCCAaACTCAGGGTCACATTC |
| | TGTCCTTCCTTTCTGAACAaTTGACGAAGCCTGACCACTCGTACAGCGTTaCCCTATAGTGAGTCGT |
| | ATTACCGGCC |
| | GCCCCATCATGTGCCTTTCcTTGACGAAGCCTGACCATGAATTCTCGCCAaACCAGTACATGGCATC |
| | GTGGTTCAAATTCTGTTCAaTTGACGAAGCCTGACCACTCGTACAGCGTTaCCCTATAGTGAGTCGT |
| | ATTACCGGCC |
| | GCCCCATCATGTGCCTTTCcTTGACGAAGCCTGACCATGAATTCTCGCCAaTAGATCAATCTCAGCC |
| | CTTGCCCTGGGTCCTGTCGaTTGACGAAGCCTGACCACTCGTACAGCGTTaCCCTATAGTGAGTCGT |
| | ATTACCGGCC |
| | GCCCCATCATGTGCCTTTCcTTGACGAAGCCTGACCATGAATTCTCGCCAaATCTCCTTTCTGAAAGT |
| | CATTTACTATCTGTGAGTaTTGACGAAGCCTGACCACTCGTACAGCGTTaCCCTATAGTGAGTCGTAT |
| | TACCGGCC |
| | GCCCCATCATGTGCCTTTCcTTGACGAAGCCTGACCATGAATTCTCGCCAaCCTTCTTCTCCCGAGA |
| | GACGCTGTACCCTTCAGCTaTTGACGAAGCCTGACCACTCGTACAGCGTTaCCCTATAGTGAGTCGT |
| | ATTACCGGCC |
| | GCCCCATCATGTGCCTTTCcTTGACGAAGCCTGACCATGAATTCTCGCCAaTTCTTTTGGGCCGATG |
| | TCACAGTGAGAGGAAAGGAaTTGACGAAGCCTGACCACTCGTACAGCGTTaCCCTATAGTGAGTC |
| | GTATTACCGGCC |
| TRBJ2-7 probe: | GCCCCATCATGTGCCTTTCcTTGACGAAGCCTGACAGCTCGGCCTGTGATaTTGACGAAGCCTGAC |
| | AGCTCGGCCTGTGATaGAGCCTGGTGCCCGGCCCGAAGTACTGCTCGTAGGaTTGACGAAGCCTG |
| | ACAATTGCATCTTCGCGaTTGACGAAGCCTGACAATTGCATCTTCGCGaCCCTATAGTGAGTCGTAT |
| | TACCGGCC |

### REFERENCES

The following references are incorporated by their entirety.
Groenen, P. J. T. A., van den Brand, M., Kroeze, L. I., Amir, A. L., and Hebeda, K. M. (2023). Read the clonotype: Next-generation sequencing-based lymphocyte clonality analysis and perspectives for application in pathology. Frontiers in Oncology 13.
Tembhare, P., Yuan, C. M., Xi, L., Morris, J. C., Liewehr, D., Venzon, D., Jani, J. E., Raffeld, M., and Stetler-Stevenson, M. (2011). Flow Cytometric Immunophenotypic Assessment of T-Cell Clonality by Vβ Repertoire Analysis. Am J Clin Pathol 135, 890-900. 10.1309/AJCPV2D 1DDSGJDBW.
Capone, M., Peruzzi, B., Palterer, B., Bencini, S., Sanna, A., Puccini, B., Nassi, L., Salvadori, B., Statello, M., Carraresi, A., et al. (2022). Rapid evaluation of T cell clonality in the diagnostic work-up of mature T cell neoplasms: TRBC1-based flow cytometric assay experience. Translational Oncology 26, 101552. 10.1016/j.tranon.2022.101552.
Boyd, S. D., Marshall, E. L., Merker, J. D., Maniar, J. M., Zhang, L. N., Sahaf, B., Jones, C. D., Simen, B. B., Hanczaruk, B., Nguyen, K. D., et al. (2009). Measurement and clinical monitoring of human lymphocyte clonality by massively parallel VDJ pyrosequencing. Sci Transl Med 1, 12ra23.
Mahe, E., Pugh, T., and Kamel-Reid, S. (2018). T cell clonality assessment: past, present and future. Journal of Clinical Pathology 71, 195-200. 10.1136/jclinpath-2017-204761.
Murphy, Kenneth, J., and Weaver, Casey (2017). Janeway's Immunobiology 9th ed. (Garland Science/Taylor & Francis).
Schatz, D. G., and Ji, Y. (2011). Recombination centres and the orchestration of V(D)J recombination. Nat Rev Immunol 11, 251-263. 10.1038/nri2941.
Garcia, K. C., and Adams, E. J. (2005). How the T Cell Receptor Sees Antigen-A Structural View. Cell 122, 333-336. 10.1016/j.cell.2005.07.015.
Flug, F., Pelicci, P. G., Bonetti, F., Knowles, D. M., and Dalla-Favera, R. (1985). T-cell receptor gene rearrangements as markers of lineage and clonality in T-cell neoplasms. Proc Natl Acad Sci USA 82, 3460-3464. 10.1073/pnas.82.10.3460.
Bertness, V., Kirsch, I., Hollis, G., Johnson, B., and Bunn, P. A. (1985). T-cell receptor gene rearrangements as clinical markers of human T-cell lymphomas. N Engl J Med 313, 534-538. 10.1056/NEJM198508293130902.
van Eden, W., Holoshitz, J., Nevo, Z., Frenkel, A., Klajman, A., and Cohen, I. R. (1985). Arthritis induced by a T-lymphocyte clone that responds to Mycobacterium tuberculosis and to cartilage proteoglycans. Proc Natl Acad Sci USA 82, 5117-5120. 10.1073/pnas.82.15.5117.
Londei, M., Bottazzo, G. F., and Feldmann, M. (1985). Human T-Cell Clones from Autoimmune Thyroid Glands: Specific Recognition of Autologous Thyroid Cells. Science 228, 85-89. 10.1126/science.3871967.
French, L. E., Lessin, S. R., Addya, K., Denardo, B., Margolis, D. J., Leonard, D. G., and Rook, A. H. (2001). Identification of clonal T cells in the blood of patients with systemic sclerosis: positive correlation with response to photopheresis. Arch Dermatol 137, 1309-1313. 10.1001/archderm.137.10.1309.
Langerak, A. W., Groenen, P. J. T. A., Bruggemann, M., Beldjord, K., Bellan, C., Bonello, L., Boone, E., Carter, G. I., Catherwood, M., Davi, F., et al. (2012). EuroClonality/BIOMED-2 guidelines for interpretation and reporting of Ig/TCR clonality testing in suspected lymphoproliferations. Leukemia 26, 2159-2171. 10.1038/leu.2012.246.
van Dongen, J. J. M., Langerak, A. W., Bruggemann, M., Evans, P. a. S., Hummel, M., Lavender, F. L., Delabesse, E., Davi, F., Schuuring, E., Garcia-Sanz, R., et al. (2003). Design and standardization of PCR primers and protocols for detection of clonal immunoglobulin and T-cell receptor gene recombinations in suspect lymphoproliferations: report of the BIOMED-2 Concerted Action BMH4-CT98-3936. Leukemia 17, 2257-2317. 10.1038/sj.leu.2403202.
Bourguin, A., Tung, R., Galili, N., and Sklar, J. (1990). Rapid, nonradioactive detection of clonal T-cell receptor gene rearrangements in lymphoid neoplasms. Proc Natl Acad Sci USA 87, 8536-8540. 10.1073/pnas.87.21.8536.
Syrykh, C., Gorez, P., Pericart, S., Grand, D., Escudie, F., Cabarrou, B., Oberic, L., Ysebaert, L., Lamant, L., Laurent, C., et al. (2021). Molecular diagnosis of T-cell lymphoma: a correlative study of PCR-based T-cell clonality assessment and targeted NGS. Blood Advances 5, 4590-4593. 10.1182/bloodadvances.2021005249.
Tan, B. T., Warnke, R. A., and Arber, D. A. (2006). The frequency of B- and T-cell gene rearrangements and epstein-barr virus in T-cell lymphomas: a comparison between angioimmunoblastic T-cell lymphoma and peripheral T-cell lymphoma, unspecified with and without associated B-cell proliferations. J Mol Diagn 8, 466-475; quiz 527. 10.2353/jmoldx.2006.060016.
Brüggemann, M., Kotrovi, M., Knecht, H., Bartram, J., Boudjogrha, M., Bystry, V., Fazio, G., Frŏnková, E., Giraud, M., Grioni, A., et al. (2019). Standardized next-generation sequencing of immunoglobulin and T-cell receptor gene recombinations for MRD marker identification in acute lymphoblastic leukaemia; a EuroClonality-NGS validation study. Leukemia 33, 2241-2253. 10.1038/s41375-019-0496-7.
Meysman, P., Barton, J., Bravi, B., Cohen-Lavi, L., Karnaukhov, V., Lilleskov, E., Montemurro, A., Nielsen, M., Mora, T., Pereira, P., et al. (2023). Benchmarking solutions to the T-cell receptor epitope prediction problem: IMMREP22 workshop report. ImmunoInformatics 9, 100024. 10.1016/j.immuno.2023.100024.
Liu, S., Iorgulescu, J. B., Li, S., Borji, M., Barrera-Lopez, I. A., Shanmugam, V., Lyu, H., Morriss, J. W., Garcia, Z. N., Murray, E., et al. (2022). Spatial maps of T cell receptors and transcriptomes reveal distinct immune niches and interactions in the adaptive immune response. Immunity 55, 1940-1952.e5. 10.1016/j.immuni.2022.09.002.
Sudmeier, L. J., Hoang, K. B., Nduom, E. K., Wieland, A., Neill, S. G., Schniederjan, M. J., Ramalingam, S. S., Olson, J. J., Ahmed, R., and Hudson, W. H. (2022). Distinct phenotypic states and spatial distribution of CD8+ T cell clonotypes in human brain metastases. Cell Reports Medicine 3, 100620. 10.1016/j.xcrm.2022.100620.
Hudson, W. H., and Sudmeier, L. J. (2022). Localization of T cell clonotypes using the Visium spatial transcriptomics platform. STAR Protocols 3, 101391. 10.1016/j.xpro.2022.101391.
Williams, C. G., Lee, H. J., Asatsuma, T., Vento-Tormo, R., and Haque, A. (2022). An introduction to spatial transcriptomics for biomedical research. Genome Medicine 14, 68. 10.1186/s13073-022-01075-1.
Lubeck, E., and Cai, L. (2012). Single-cell systems biology by super-resolution imaging and combinatorial labeling. Nat Methods 9, 743-748. 10.1038/nmeth.2069.
Lubeck, E., Coskun, A. F., Zhiyentayev, T., Ahmad, M., and Cai, L. (2014). Single-cell in situ RNA profiling by sequential hybridization. Nat Methods 11, 360-361. 10.1038/nmeth.2892.
Chen, K. H., Boettiger, A. N., Moffitt, J. R., Wang, S., and Zhuang, X. (2015). Spatially resolved, highly multiplexed RNA profiling in single cells. Science 348, aaa6090. 10.1126/science.aaa6090.
Eng, C. H. L., Shah, S., Thomassie, J., and Cai, L. (2017). Profiling the transcriptome with RNA SPOTs. Nat Methods 14, 1153-1155. 10.1038/nmeth.4500.
Eng, C. H. L., Lawson, M., Zhu, Q., Dries, R., Koulena, N., Takei, Y., Yun, J., Cronin, C., Karp, C., Yuan, G. C., et al. (2019). Transcriptome-scale super-resolved imaging in tissues by RNA seqFISH+. Nature 568, 235-239. 10.1038/s41586-019-1049-y.
Femino, A. M., Fay, F. S., Fogarty, K., and Singer, R. H. (1998). Visualization of single RNA transcripts in situ. Science 280, 585-590. 10.1126/science.280.5363.585.
Raj, A., van den Bogaard, P., Rifkin, S. A., van Oudenaarden, A., and Tyagi, S. (2008). Imaging individual mRNA molecules using multiple singly labeled probes. Nat Methods 5, 877-879. 10.1038/nmeth.1253.
Lehner, P. J., Wang, E. C., Moss, P. A., Williams, S., Platt, K., Friedman, S. M., Bell, J I., and Borysiewicz, L. K. (1995). Human HLA-A0201-restricted cytotoxic T lymphocyte recognition of influenza A is dominated by T cells bearing the V beta 17 gene segment. J Exp Med 181, 79-91. 10.1084/jem.181.1.79.
Stewart-Jones, G. B. E., McMichael, A. J., Bell, J. I., Stuart, D. I., and Jones, E. Y. (2003). A structural basis for immunodominant human T cell receptor recognition. Nat Immunol 4, 657-663. 10.1038/ni942.
Valkenburg, S. A., Josephs, T. M., Clemens, E. B., Grant, E. J., Nguyen, T. H. O., Wang, G. C., Price, D. A., Miller, A., Tong, S. Y. C., Thomas, P. G., et al. (2016). Molecular basis for universal HLA-A*0201-restricted CD8+ T-cell immunity against influenza viruses. Proc Natl Acad Sci USA 113, 4440-4445. 10.1073/pnas.1603106113.
Yang, X., Chen, G., Weng, N., and Mariuzza, R. A. (2017). Structural basis for clonal diversity of the human T-cell response to a dominant influenza virus epitope. J Biol Chem 292, 18618-18627. 10.1074/jbc. M117.810382.
Arden, B., Clark, S. P., Kabelitz, D., and Mak, T. W. (1995). Human T-cell receptor variable gene segment families. Immunogenetics 42, 455-500. 10.1007/BF00172176.
Dirks, R. M., and Pierce, N. A. (2004). Triggered amplification by hybridization chain reaction. Proc Natl Acad Sci USA 101, 15275-15278. 10.1073/pnas.0407024101.
Shah, S., Lubeck, E., Schwarzkopf, M., He, T. F., Greenbaum, A., Sohn, C. H., Lignell, A., Choi, H. M. T., Gradinaru, V., Pierce, N. A., et al. (2016). Single-molecule RNA detection at depth by hybridization chain reaction and tissue hydrogel embedding and clearing. Development 143, 2862-2867. 10.1242/dev.138560.
Rouhanifard, S. H., Mellis, I. A., Dunagin, M., Bayatpour, S., Jiang, C. L., Dardani, I., Symmons, O., Emert, B., Torre, E., Cote, A., et al. (2019). ClampFISH detects individual nucleic acid molecules using click chemistry-based amplification. Nat Biotechnol 37, 84-89. 10.1038/nbt.4286.
Dardani, I., Emert, B. L., Goyal, Y., Jiang, C. L., Kaur, A., Lee, J., Rouhanifard, S. H., Alicea, G. M., Fane, M. E., Xiao, M., et al. (2022). ClampFISH 2.0 enables rapid, scalable amplified RNA detection in situ. Nat Methods 19, 1403-1410. 10.1038/s41592-022-01653-6.
Chauveau, A., Pirgova, G., Cheng, H. W., De Martin, A., Zhou, F. Y., Wideman, S., Rittscher, J., Ludewig, B., and Arnon, T. I. (2020). Visualization of T Cell Migration in the Spleen Reveals a Network of Perivascular Pathways that Guide Entry into T Zones. Immunity 52, 794-807.e7. 10.1016/j.immuni.2020.03.010.
Steiniger, B. S. (2015). Human spleen microanatomy: why mice do not suffice. Immunology 145, 334-346. 10.1111/imm.12469.
Lewis, S. M., Williams, A., and Eisenbarth, S. C. (2019). Structure-function of the immune system in the spleen. Sci Immunol 4, eaau6085. 10.1126/sciimmunol.aau6085.
Frisoli, M. L., Essien, K., and Harris, J. E. (2020). Vitiligo: Mechanisms of Pathogenesis and Treatment. Annu Rev Immunol 38, 621-648. 10.1146/annurev-immunol-100919-023531.
Yancey, W. E. (2010). Expected Number of Random Duplications Within or Between Lists. In JSM Proceedings (American Statistical Association), pp. 2938-2946.
Snyder, M. P., Lin, S., Posgai, A., Atkinson, M., Regev, A., Rood, J., Rozenblatt-Rosen, O., Gaffney, L., Hupalowska, A., Satija, R., et al. (2019). The human body at cellular resolution: the NIH Human Biomolecular Atlas Program. Nature 574, 187-192. 10.1038/s41586-019-1629-x.
Manso, T., Folch, G., Giudicelli, V., Jabado-Michaloud, J., Kushwaha, A., Nguefack Ngoune, V., Georga, M., Papadaki, A., Debbagh, C., Pegorier, P., et al. (2022). IMGT@databases, related tools and web resources through three main axes of research and development. Nucleic Acids Res 50, D1262-D1272. 10.1093/nar/gkab1136.
Ma, L., Yang, L., Bin Shi, He, X., Peng, A., Li, Y., Zhang, T., Sun, S., Ma, R., and Yao, X. (2016). Analyzing the CDR3 Repertoire with respect to TCR-Beta Chain V-D-J and V-J Rearrangements in Peripheral T Cells using HTS. Sci Rep 6, 29544. 10.1038/srep29544.
Riding, R. L., and Harris, J. E. (2019). The role of Memory CD8+ T cells in Human Vitiligo. J Immunol 203, 11-19. 10.4049/jimmunol.1900027.
Cheuk, S., Schlums, H., Gallais Serezal, I., Martini, E., Chiang, S. C., Marquardt, N., Gibbs, A., Detlofsson, E., Introini, A., Forkel, M., et al. (2017). CD49a Expression Defines Tissue-Resident CD8+ T Cells Poised for Cytotoxic Function in Human Skin. Immunity 46, 287-300. 10.1016/j.immuni.2017.01.009.
Bradley, L. (2023). astropy/photutils: 1.8.0. 10.5281/zenodo.7946442.
Stetson, P. B. (1987). DAOPHOT: A COMPUTER PROGRAM FOR CROWDED-FIELD STELLAR PHOTOMETRY. PASP 99, 191. 10.1086/131977.
Stringer, C., Wang, T., Michaelos, M., and Pachitariu, M. (2021). Cellpose: a generalist algorithm for cellular segmentation. Nat Methods 18, 100-106. 10.1038/s41592-020-01018-x.
Waskom, M. L. (2021). seaborn: statistical data visualization. Journal of Open Source Software 6, 3021. 10.21105/joss.03021.
Peng, K., Moore, J., Vahed, M., Brito, J., Kao, G., Burkhardt, A. M., Alachkar, H., and Mangul, S. (2022). pyTCR: A comprehensive and scalable solution for TCR-Seq data analysis to facilitate reproducibility and rigor of immunogenomics research. Frontiers in Immunology 13, 954078.
Gu, Z., Gu, L., Eils, R., Schlesner, M., and Brors, B. (2014). circlize implements and enhances circular visualization in R. Bioinformatics 30, 2811-2812. 10.1093/bioinformatics/btu393.
Bois, J. (2022). justinbois/iqplot: 0.3.2. 10.22002/D1.20286.
Hu, X. et al. A Simple and Efficient Method for Preparing Cell Slides and Staining without Using Cytocentrifuge and Cytoclips. International Journal of Cell Biology. Nov. 17, 2015.
PCT/US2022/021826 Linked amplification tethered with exponential radiance. International filing date: Mar. 24, 2022
International PCT Patent Application No. PCT/US2014/036258, filed Apr. 30, 2014, and titled MULTIPLEX LABELING OF MOLECULES BY SEQUENTIAL HYBRIDIZATION BARCODING.
International PCT Patent Application No. PCT/US2018/064616, filed Dec. 7, 2018, and titled MULTIPLEX LABELING OF MOLECULES Publication Classification BY SEQUENTIAL HYBRIDIZATION BARCODING WITH RAPID SWITCHING AND REHYBRIDIZATION OF PROBES.
International PCT Patent Application No. PCT/US2017/044994, filed Aug. 1, 2017, and titled SEQUENTIAL PROBING OF MOLECULAR TARGETS BASED ON PSEUDO—COLOR BARCODES WITH EMBEDDED ERROR CORRECTION MECHANISM.
International PCT Patent Application No. PCT/US2022/024494, filed Apr. 12, 2022, and titled HIGH-RESOLUTION WHOLE GENOME IMAGING BY NUCLEIC ACID LOCUS AND BLOCK CODING.
International PCT Patent Application No. PCT/US2022/032736, filed Jun. 8, 2022, and titled RATIOMETRIC SYMBOLS AND SEQUENTIAL CODING FOR MULTIPLEXED FISH.
International PCT Patent Application No. PCT/US2022/053995, filed Dec. 23, 2022, and titled SUPPRESSION OF NON-SPECIFIC SIGNALS BY EXONUCLEASES IN FISH EXPERIMENT.
International PCT Patent Application No. PCT/US2022/051737, filed Dec. 2, 2022, and titled METHOD OF MAPPING SPATIAL DISTRIBUTIONS OF CELLULAR COMPONENTS.
International PCT Patent Application No. PCT/US2022/017757, filed Feb. 24, 2022, and titled MULTIPLEXING OF EXPERIMENTAL CONDITIONS AND SAMPLES IN SPATIAL GENOMICS.
International PCT Patent Application No. PCT/US2025/015646, filed Feb. 12, 2025, and titled SUPER-RESOLVED OBJECT DETECTION WITH SPATIAL GENOMICS.
International PCT Patent Application No. PCT/US2026/010077, filed Jan. 2, 2026, and titled READ-AND-ANCHOR SPLIT PROBE STRATEGY FOR HIGH-SPECIFICITY MOLECULAR PROFILING.

We claim:

1. A method, comprising steps of:
(a) contacting a cell population with a plurality of primary probes, so that the plurality of primary probes comprises:
(i) a first primary probe, that interacts with a first gene segment;
(ii) optionally, a second primary probe, that interacts with a second gene segment; and
(iii) optionally, a third primary probe, that interacts with a third gene segment; wherein the first, second, and third primary probes are different from each other; and
(b) imaging the cell population after the contacting step so that interaction of the probes with their gene segments are detected.

2. The method of claim 1, wherein the plurality of primary probes comprises the first primary probe and the second primary probe.

3. The method of claim 1, wherein the plurality of primary probes comprises the first primary probe, the second primary probe, and the third primary probe.

4. The method of any of claims 1 to 3, wherein the method further comprises:
(c) optionally, contacting a plurality of secondary probes to the primary probes before step (b), wherein each secondary probe comprises one or more tertiary probe binding sites and/or one or more readout probe binding sites, and wherein each secondary probe in the plurality of the secondary probes interacts with either the first primary probe, second primary probe, and/or third primary probe;
(d) optionally, contacting a plurality of tertiary probes to the secondary probes before step (b), wherein each tertiary probe comprises one or more quaternary probe binding sites and/or one or more readout probe binding sites, and wherein each tertiary probe in the plurality of the tertiary probes interacts with one of the secondary probes in the plurality of secondary probes;
(e) optionally, contacting a plurality of quaternary probes to the tertiary probes before step (b), wherein each quaternary probe comprises one or more readout probe binding sites, and wherein each quaternary probe in the plurality of the quaternary probes interacts with one of the tertiary probes in the plurality of tertiary probes; and
(f). contacting the primary probes, secondary probes, tertiary probes, and/or quaternary probes with one or more readout probes before step (b).

5. The method of claim 4, further comprising:
(g) optionally, stabilizing the primary, secondary, tertiary, and/or quaternary probes during or after each contacting step.

6. The method of claim 4, further comprising:
(h) optionally, washing the cell population to remove the primary probes, secondary probes, tertiary probes, quaternary probes, and/or readout probes;
(i) sequencing nucleic acid sequences in proximity to the gene segments;
(k) correlating the nucleic acid sequences to detected gene segments in the cells of the cell population.

7. The method of claim 4, wherein the probes barcode the gene segments in the cells of the cell population.

8. The method of claim 1, wherein the first, second, and/or third gene segments are in the cells of the cell population.

9. The method of claim 1, wherein the first, second and/or third primary probes comprise one or more secondary probe binding sites and/or one or more readout probe binding sites.

10. The method of claim 1, wherein the cell population comprises lymphocytes.

11. The method of claim 10, wherein the lymphocytes are T-lymphocytes and B-lymphocytes.

12. The method of claim 10, wherein the lymphocytes are T-lymphocytes.

13. The method of claim 10, wherein the lymphocytes are B-lymphocytes.

14. The method of claim 6, wherein the cell population is sequenced.

15. The method of claim 14, wherein the sequencing is by sequencing-by-ligation.

16. The method of claim 14, wherein the sequencing is by sequencing-by synthesis.

17. The method of claim 14, wherein the sequencing is by long-read sequencing.

18. The method of claim 1, wherein each gene segment encode part of a T cell receptor gene or immunoglobulin (Ig) protein.

19. The method of claim 1, wherein the gene segments are variable (V) joining (J), and/or diversity (D) segments.

20. The method of claim 19, wherein the gene segments are in the genome of the cells of the cell population.

21. The method of claim 19, wherein the gene segments are in one or more RNA transcripts of the cells of the cell population.

22. The method of claim 19, wherein the first gene segment is a variable (V) or joining segment (J).

23. The method of claim 19, wherein the second gene segment is a variable (V) or joining segment (J).

24. The method of claim 19, wherein the first primary probe interacts with the V segment, and wherein the second primary probe interacts with the J segment.

25. The method of claim 19, wherein the first primary probe interacts with the J segment, and wherein the second primary probe interacts with the V segment.

26. The method of any of the preceding claims, wherein each primary probe comprises a nucleic acid sequence complementary to a gene segment.

27. The method of claim 26, wherein the nucleic acid sequence is 60%, 65%, 70%, 75%, 80%, 85%, 90%, 91%, 92%, 93%, 94%, 95%, 96%, 97%, 98%, 99%, or 100% complementary to the gene segment.

28. The method of claim 26, wherein the complementary sequence is between 5-1000 nucleotides long.

29. The method of claim 7, wherein the barcoded cells establish lineage information for the cells of the cell population.

30. The method of claim 29, wherein the lineage information indicates changes to variable (V), joining (J), or diversity (D) segments in the cells of the cell population.

31. The method of any of the previous claims, wherein the cell population is a tissue sample or a section of the tissue sample.

32. The method of claim 31, wherein the method further comprises:
(l) optionally, sectioning the tissue into one or more tissue sections before contacting the cell population with a plurality of primary probes in step (a);
(m) forming a map of one of the tissue sections, wherein the map correlates the nucleic acid read sequences to their detected gene segments in the cells of the tissue section; and
(m) assembling a global map comprising two or more maps of tissue sections.

33. The method of claim 10, wherein the method further comprises a histological analysis of the lymphocyte population.

34. The method of claim 10, further comprising sorting the lymphocyte population to separate lymphocytes with different barcodes.

35. The method of claim 34, wherein the lymphocyte population is genomically sequenced.

36. The method of claim 34, wherein the sorted lymphocyte population is genomically sequenced.

37. The method of claim 36, wherein the genomic sequencing sequences DNA, mRNA, or any combination thereof.

38. The method of claim 1, further comprising determining a structure for a T cell receptor and/or an immunoglobulin protein.

39. The method of any of the preceding claims wherein a readout probe hybridizes to one or more of the primary, secondary, tertiary, and/or quaternary probes.

40. The method of any one of the preceding claims, wherein the readout probes comprise oligonucleotides with detectable moieties.

41. The method of any one of the preceding claims, wherein the readout probes comprise oligonucleotides with the same sequence.

42. The method of any one of the preceding claims, wherein the readout probes comprise oligonucleotides with different sequences.

43. The method of any one of the preceding claims, wherein the readout probes comprise detectable moieties that are the same.

44. The method of any one of the preceding claims, wherein the readout probes comprise detectable moieties that are different.

45. The method of claim 4, wherein the one or more readout probes are sequencing probes.

46. The method of claim 45, wherein the sequencing probes are sequenced by sequencing-by-ligation.

47. The method of claim 45, wherein the sequencing probes are sequenced by sequencing-by synthesis.

48. The method of claim 45, wherein the sequencing probes are sequenced by long-read sequencing.

49. The method of any of the preceding claims, wherein the cell population is washed after each step.

50. The method of claim 49, wherein the cell population is washed with a buffer that removes non-specific hybridization reactions.

51. The method of any of the previous claims, wherein super-resolution microscopy is used to image the barcode.

52. The method of any of the preceding claims wherein the gene segments of T cell receptors or immunoglobulins are localized in the cells of the cell population.

53. The method of claim 52, wherein the gene segments are V, J, and/or D pairings detected by hybridization of probes and imaging.

54. The method of claim 52, wherein the gene segments are V and J pairings detected by hybridization of probes and imaging.

55. The method of any of the preceding claims wherein the gene segments of T cell receptors or immunoglobulins are barcoded in the cells of the cell population.

56. The method of claim 55, wherein the gene segments are V, J, and/or D pairings detected by hybridization of probes and imaging.

57. The method of claim 55, wherein the gene segments are V and J pairings detected by hybridization of probes and imaging.
